(19) United States
(12) Patent Application Publication
(10) Pub. No.: US 2014/0073519 A1
(43) Pub. Date: Mar. 13, 2014

(54) LUNG CANCER-RELEVANT HUMAN EMBRYONIC STEM CELL SIGNATURE

(71) Applicants: Ronald G. Crystal, New York, NY (US); Renat Shaykhiev, New York, NY (US)

(72) Inventors: Ronald G. Crystal, New York, NY (US); Renat Shaykhiev, New York, NY (US)

(73) Assignee: Cornell University, Ithaca, NY (US)

(21) Appl. No.: 14/002,871

(22) PCT Filed: Mar. 5, 2012
(86) PCT No.: PCT/US12/27744

§ 371 Date: Nov. 19, 2013

Related U.S. Application Data
(60) Provisional application No. 61/448,948, filed on Mar. 3, 2011.

(51) Int. Cl. C12Q 1/68

(52) U.S. Cl. CPC C12Q 1/6886
USPC 506/9

(57) ABSTRACT

The invention provides a method of detecting cancer, a progression of cancer, or a predisposition to cancer in a human, comprising (a) obtaining a sample of airway basal cells from the human, and (b) analyzing the sample to determine expression of one or more hESC-signature genes, wherein the expression or lack of expression of the one or more hESC-signature genes is indicative of a presence or absence of cancer, a progression of cancer, or a predisposition to cancer in the human. The invention also provides an in vitro model for lung cancer, comprising airway basal cells that express one or more hESC-signature genes.

### CROSS-REFERENCE TO RELATED APPLICATIONS

This patent application claims the benefit of U.S. Provisional Patent Application No. 61/448,948, filed Mar. 3, 2011, which is incorporated by reference.

### STATEMENT REGARDING FEDERALLY SPONSORED RESEARCH AND DEVELOPMENT

This invention was made with Government support under National Heart, Lung and Blood Institute Grant Number P50 HL084936 and National Center for Research Resources Grant Number UL1-RR024996. The Government has certain rights in this invention.

### BACKGROUND OF THE INVENTION

Lung cancer is the most common cause of cancer mortality in both men and women, accounting for about 28% of all cancer deaths in the United States. Lung cancer is generally classified as small cell (14%) or non-small cell (85%) for the purposes of treatment. Regardless of the subtype, the 5-year survival rate for patients with lung cancer is among the lowest of all cancers, at only 16%. The 5-year survival rate is 52% in instances when the lung cancer is detected while still localized, but only 15% of lung cancers are diagnosed at this early stage (American Cancer Society, Cancer Facts & Figures 2012, Atlanta, American Cancer Society (2012)). Early detection and diagnosis are therefore critical in reducing the morbidity and mortality associated with lung cancer.

Current lung cancer screening methods include chest x-rays, low-dose helical computed tomography (CT) scans, and pathological examinations of sputum or biopsy samples. However, these methods have not been definitively proven to improve clinical outcome, and the risks associated with these methods, including cumulative radiation exposure from multiple CT scans and unnecessary lung biopsy and surgery, have not yet been evaluated. No generally accepted screening guidelines exist at the present time (American Cancer Society, Cancer Facts & Figures 2012, Atlanta, American Cancer Society (2012)).

It is clear, therefore, that there is a strong need for additional and improved methods of screening for lung cancer.

### BRIEF SUMMARY OF THE INVENTION

The invention provides a method of detecting cancer, a progression of cancer, or a predisposition to cancer in a human, comprising (a) obtaining a sample of airway basal cells from the human, and (b) analyzing the sample to determine expression of one or more human embryonic stem cell (hESC)-signature genes, wherein the expression or lack of expression of the one or more hESC-signature genes is indicative of a presence or absence of cancer, a progression of cancer, or a predisposition to cancer in the human. The invention also provides an in vitro model for lung cancer, comprising airway basal cells that express one or more hESC-signature genes.

### BRIEF DESCRIPTION OF THE DRAWINGS

FIGS. 1A-1E show enrichment of hESC-signature genes in airway basal cells. FIG. 1A depicts the immunocytochemical verification of basal cell phenotype. After 7 days of culture of freshly isolated lung airway epithelium (LAE) cells, the cells were analyzed for expression of cytokeratin 5 (basal cell-specific marker), N-cahedrin (mesenchymal marker), mucin 5AC (secretory cell marker), and β-tubulin IV (ciliated cell marker). Scale bar=10 μm. FIG. 1B depicts the basal cell differentiation into ciliated airway epithelium on ALI. Appearance of ciliated cells was monitored by expression of β-tubulin IV weekly by immunofluorescence. Scale bar=10 μm. FIG. 1 C is a volcano plot comparing expression of hESC-signature gene probe sets in BC-NS (n=4) versus LAE-NS (n=21). FIG. 1D depicts the principal component analysis of LAE-NS (n=21) and BC-NS (n=4) based on expression of hESC-signature gene probe sets detected in LAE-NS and/or BC-NS. The samples within each group were placed in a 3 dimensional space based on the expression pattern using mean centering and scaling function; each circle represents an individual sample. The percentage contributions of the first 3 principal components (PC) to the observed variability are indicated. FIG. 1E is a heat-map of the hESC-signature gene expression changes during BC differentiation on ALI. Genes detected in at least one group were mapped and coded according to their mean normalized expression at each time point (n=3 in each group).

FIGS. 2A-2E show induction of the hESC-signature in BC-S. FIG. 2A is a pair of graphs. Left panel—volcano plot comparing expression of hESC-signature gene probe sets in LAE of healthy smokers (LAE-S; n=31) versus LAE of healthy nonsmokers (LAE-NS; n=21). Right panel—volcano plot comparing expression of hESC-signature gene probe sets in BC-S (n=4) versus BC-NS (n=4). FIG. 2B is a pair of graphs depicting principal component analysis of BC-NS (n=4) and BC-S (n=4) on all expressed gene probe sets (left panel) and hESC-signature gene probe sets (right panel). The percentage contributions of the first principal component (PC1) to the observed variabilities are indicated. FIG. 2C is an unsupervised hierarchical cluster analysis of BC-NS and BC-S based on expression of detected hESC-signature genes. FIG. 2D is a bar graph depicting fold-changes for differentially expressed hESC-signature genes in BC-S versus BC-NS determined by microarray analysis (white bars; n=4 in each group) and RNA-Seq (black bars; n=2 in each group). FIG. 2E is a bar graph depicting expression of selected hESC-signature genes in BC-NS stimulated with 2% CSE for 48 hours (n=3) compared to unstimulated cells (n=3) determined by TAQMAN™ PCR; * p<0.05; N.S.—nonsignificant; N.D.—not detected.

FIGS. 3A-3E show the relevance of the BC-S hESC-signature to lung adenocarcinoma (AdCa). FIG. 3A is a series of volcano plots comparing the expression of hESC-signature gene probe sets in human lung AdCa cells following passage in immunocompromised mice (n=4) versus each of the following groups: LAE-NS (n=21; upper left panel), LAE-S (n=31; upper right panel), BC-NS (n=4; lower left panel), and BC-S (n=4; lower right panel). FIG. 3B is a box-plot showing hESC index distribution in LAE-NS (n=21), LAE-S (n=31), BC-NS (n=4), BC-S (n=4), and primary lung AdCa (n=193). P values indicated were determined by ANOVA post-hoc with Bonferroni/Dunn correction. FIG. 3C is a graph depicting principal component analysis of all individual samples belonging to indicated groups using the list of hESC-specific genes expressed in these study groups as an input dataset. FIG. 3D is an unsupervised hierarchical clustering analysis of all individual samples belonging to indicted groups based on expression of hESC-signature genes. FIG. 3E is a graph depicting a Kaplan-Meier analysis-based estimates of overall survival of lung AdCa patients highly expressing a BC-S hESC-signature gene cluster (high expressors; n=44,) versus low expressors of these genes (n=42); p values indicated were determined by the log-rank test.

FIGS. 4A-4H are a series of graphs showing the association between BC-S hESC-signature and TP53 molecular phenotype. FIG. 4A is a box-plot showing BC-S hESC index distribution in primary AdCa divided based on smoking status (NS—nonsmokers; S—smokers) and TP53 status (WT—wild-type; *—mutation): AdCa-NS-TP53WT (n=29), AdCa-NS-TP53* (n=7), AdCa-S-TP53WT (n=95), AdCa-S-TP53* (n=36). P values indicated were determined by ANOVA post-hoc with Bonferroni/Dunn correction. FIG. 4B is a graph depicting a Spearman correlation analysis of relationship between BC-S hESC index and TP53-inactivation (TP53i) index in AdCa-S-TP53* (n=36); Spearman rank correlation coefficient (Rho) and p value indicated. FIG. 4C is a graph depicting the expression of selected BC-S hESC-signature genes in indicated TP53WT and TP53* lung cancer cell lines (n=4 for each cell line) determined by TAQMANTM PCR. FIG. 4D is a graph depicting principal component analysis of indicated groups based on expression of BC-S hESC-signature genes (upper panel) and TP53i gene signature (lower panel). FIG. 4E is a pair of volcano plots comparing expression of TP53i-signature gene probe sets in LAE-S (n=31) versus LAE-NS (n=21)—upper panel; and in BC-S (n=4) versus BC-NS (n=4). FIG. 4F is a pair a graphs depicting normalized expression of BC-S hESC-signature genes (upper panel) and TP53-inactivation signature genes (lower panel) in BC-NS (n=4) and BC-S (n=4). FIG. 4G is a graph depicting Spearman correlation analysis of relationship between BC-S hESC index and TP53-inactivation index in BC-NS (n=4) and BC-S (n=4); Spearman rank correlation coefficient (Rho) and p value indicated. FIG. 4H is a graph depicting the significant up-regulation of CDKN2A in BC-S compared to BC-NS.

FIGS. 5A-5G show overexpression of BC-S hESC-signature genes in various types of human lung cancer. FIG. 5A is a chart that sets forth mapping information based on the indicated parameters for BC-S hESC-signature genes (left cluster) and other hESC-signature genes (right cluster). Genes that meet the criteria are highlighted light grey; genes with opposite change are highlighted dark grey; genes not detectable by the given microarray platform are indicated with black boxes. Original datasets included LAE-NS (n=21), LAE-S (n=31), BC-NS (n=4), BC-S (n=4), lung AdCa cells propagated in a xenograft model (AdCa-Xeno; n=4), and primary lung AdCa (AdCa; n =193) (Chitale et al., Oncogene, 28: 2773-2783 (2009)). Independent lung cancer datasets were analyzed using ONCOMINE database, including lung AdCa datasets Landi et al. (L; n=58) (Landi et al., PLoS One, 3: e1651 (2008)), Kuner et al. (K; n=42) (Kuner et al., Lung Cancer, 63: 32-38 (2009)), Garber et al. (G; n=40) (Garber et al., Proc. Natl. Acad. Sci. USA, 98: 13784-13789 (2001)), squamous cell lung carcinoma (SCC) datasets Kuner et al. (K; n=18) (Kuner et al., Lung Cancer, 63: 32-38 (2009)), Garber et al. (G; n=13) (Garber et al., Proc. Natl. Acad. Sci. USA, 98: 13784-13789 (2001)), comparison of SCC to AdCa in datasets Kuner et al. (Kuner et al., Lung Cancer, 63: 32-38 (2009)), Bild et al. (B, SCC, n=53; AdCa, n=58) (Bild et al., Nature, 439: 353-357 (2006)), small cell lung carcinoma (SCLC; n=4) and large cell lung carcinoma (LCLC; n=4) datasets Garber et al. (G) (Garber et al., Proc. Natl. Acad. Sci. USA, 98: 13784-13789 (2001)). FIG. 5B depicts principal component analysis of LAE-NS, LAE-S, BC-NS, BC-S, independent AdCa and SCC datasets Kuner et al. (Kuner et al., Lung Cancer, 63: 32-38 (2009)), and hESC from datasets Avery et al. (n=3) (Avery et al., Stem Cells Dev., 17: 1195-1205 (2008)) and Denis et al. (GSE8590; n=2) (Denis et al., Stem Cells Dev., 20(8): 1395-1409 (2011)). FIG. 5C depicts that, when the entire hESC-signature was used as an input dataset, a subset of AdCa samples and the majority of majority of SCC shared with BC-S, but not BC-NS, similar distribution with a notable shift toward hESC. FIG. 5D depicts that further restriction of the analysis to the 15-gene BC-S hESC-signature revealed similarity of the SCC samples and a subset of the AdCa samples to both BC-S and hESC. FIG. 5E depicts that the spatial pattern was effectively reproduced using the dataset containing 6 co-expressed prognostically relevant BC-S hESC-signature genes. FIG. 5F depicts that the spacial pattern was not effectively reproduced using the dataset containing the non-BC-S hESC-signature genes. FIG. 5G depicts that SCC and a subset of AdCa samples clustered together with BC-S and hESC based on expression of the TP53-inactivation signature.

FIGS. 6A and 6B are two parts of a chart that shows the characterization of hESC-signature gene expression in airway basal cells of healthy nonsmokers and healthy smokers compared to lung adenocarcinoma.

FIG. 7 is a chart that shows the characterization of hESC-specific gene expression in airway basal cells by RNA-sequencing (RNA-Seq).

FIG. 8 is a chart that shows the characterization of hESC-specific gene expression in primary human lung adenocarcinomas as compared to all other study groups.

FIGS. 9A and 9B are graphs that show hESC-signature gene expression in the LAE and basal cells of healthy nonsmokers. FIG. 9A pertains to detection frequency. Ordinate represents the percent of subjects in each group expressing a given gene (Affymetrix present detection call). Abcissa—40 hESC-signature genes identified by Assou et al. (Assou et al., Stem Cells, 25: 961-973 (2007)) listed in alphabetic order. Shown is data for LAE-NS (n=21) and BCNS (n=4). FIG. 9B pertains to normalized expression. Ordinate represents average gene expression values normalized per array for LAE-NS (n=21) and BC-NS (n=4). Abscissa represents hESC-signature genes. The gene descriptions and detailed expression data for FIGS. 9A and 9B are set forth in FIGS. 6A and 6B.

FIG. 10 is a bar graph that depicts data comparing the mean normalized expression levels for 10 known housekeeping genes in BC-NS (n=4) and BC-S (n=4). In all comparisons, the difference between the groups is not significant (p>0.05; no Benjamini-Hochberg correction applied to increase the sensitivity of the test). The full gene names: actin, beta (ACTB), Rho GDP dissociation inhibitor (GDI) alpha (ARHGDIA), ATPase, H+ transporting, lysosomal 13 kDa, V1 subunit G isoform 1 (ATP6V1G1), endosulfine alpha (ENSA), glyceraldehyde-3-phosphate dehydrogenase (GAPDH), lactate dehydrogenase A (LDHA), ribosomal protein S18 (RPS18), ribosomal protein L19 (RPL19), ribosomal protein S27a (RPS27A), ribosomal protein L32 (RPL32).

FIGS. 11A and 11B are pairs of graphs that show principal component analysis of (left panels) LAE-NS (n=21), LAE-S (n=31) and (right panels) BC-S (n=4), BC-S (n=4) based on expression of all gene probe sets (FIG. 11A) and hESC-signature gene probe sets (FIG. 11B). The percentage contributions of the first 3 principal components (PC1-3) to the observed variabilities are indicated.

FIGS. 12A and 12B show analysis of hESC-signature gene expression in airway basal cells by massively parallel RNA-Sequencing (RNA-Seq). FIG. 12A is a Venn diagram showing overlap of hESC-signature genes detected in basal cells by Affymetrix HG-U133 Plus 2 microarray (n=21) and by RNA-Seq (n=31). Other areas represent hESC-signature genes up-regulated in BC-S (n=4 microarray analysis; n=2 RNA-Seq) versus BC-NS (n=4 microarray analysis; n=2 RNA-Seq) as determined by microarray (n=12) and RNA-Seq (n=14), respectively. Merged area represents 11 hESC-signature genes up-regulated in BC-S versus BC-NS as determined by both microarray and RNA-Seq. FIG. 12B is a visualization of RNA-Seq reads for 6 hESC-signature gene examples for BC-NS (n=2) and BC-S (n=2) using Partek Genomics Suite (Bowtie alignment algorithm v0.11.3). Horizontal tracks represent gene structure with known exons (Ex) mapped according to their physical position. The y-axis corresponds to number of reads mapping to each exon for each gene in each individual sample, i.e., reads for BC-NS and for BC-S. Cumulative expression level of each gene in each sample (determined as reads per kilobase of exon model per million mapped reads, RPKM) is shown below the label for the corresponding sample on the left of each plot. For the CHEK2 gene, exons 9, 10, and 14, containing no or barely detected reads without difference between the study groups, are not shown.

FIG. 13 is a chart that shows clinical characteristics of lung adenocarcinoma phenotypes identified based on expression of the 6-gene BC-S hESC-signature.

### DETAILED DESCRIPTION OF THE INVENTION

The invention provides a method of detecting cancer, a progression of cancer, or a predisposition to cancer in a human.

A number of cancers have been shown to express some of the 40 genes (Assou et al., Stem Cells, 25: 961-973 (2007)) specifically expressed in human embryonic stem cells (hESC-signature genes). For example, Ben-Porath et al. have shown that histologically poorly differentiated breast cancers, glioblastomas, and bladder carcinomas display preferential overexpression of genes normally enriched in embryonic stem cells, combined with underexpression of Polycomb-regulated genes (Ben-Porath et al., Nat. Genet., 40: 499-507 (2008)), and Wong et al. have shown that an embryonic stem cell-like transcriptional program is activated in diverse human epithelial cancers and strongly predicts metastasis and death (Wong et al., Cell. Stem Cells, 2: 333-344 (2008)).

Several additional studies have focused more specifically on the expression of such genes in lung cancers. In particular, Hassan et al. have shown that increased expression of the embryonic stem cell gene set and decreased expression of Polycomb target gene set identified poorly-differentiated lung adenocarcinoma, but not lung squamous cell carcinoma (Hassan et al., Clin. Cancer Res., 15(20): 6386-6390 (2009)), and Stevenson et al. have shown that lung adenocarcinomas that share a common gene expression pattern with normal human embryonic stem cells were associated with decreased survival, increased biological complexity, and increased likelihood of resistance to cisplatin (Stevenson et al., Clin. Cancer Res., 15(24): 7553-7561 (2009)). However, none of these studies have identified the cellular origins of early molecular changes in the airway epithelium relevant to the development of lung cancer.

The lung airway epithelium (LAE) comprises basal, ciliated, secretory, and columnar cells. The invention is predicated, at least in part, on the discovery that (a) certain hESC-signature genes are differentially expressed between the LAE in healthy nonsmokers (LAE-NS) and isolated basal cells in healthy nonsmokers (BC-NS) (Example 9); (b) the expression of hESC-signature genes in the LAE of healthy smokers (LAE-S) does not differ significantly from that of LAE-NS, but basal cells of healthy smokers (BC-S) exhibit a broad up-regulation of hESC-signature genes (BC-S hESC-signature) (Example 10); (c) the BC-S hESC-signature contributes to the hESC-like phenotype of lung adenocarcinoma (Example 11); (d) the BC-S hESC-signature predicts aggressive clinical phenotype in lung adenocarcinoma (Example 12); (e) the BC-S hESC-signature is associated with a TP53-inactivation molecular phenotype (Example 13); and (f) the BC-S hESC-signature contributes to the hESC-like phenotype of various types of lung cancer (Example 14).

The inventive method of detecting cancer, a progression of cancer, or a predisposition to cancer in a human comprises (a) obtaining a sample of airway basal cells from the human, and (b) analyzing the sample to determine expression of one or more hESC-signature genes, wherein the expression or lack of expression of the one or more hESC-signature genes is indicative of a presence or absence of cancer, a progression of cancer, or a predisposition to cancer in the human.

The sample can be obtained by any suitable method. Suitable methods of obtaining the sample include flexible bronchoscopy and biopsy.

The sample can be analyzed to determine expression of one or more of the hESC-signature genes by any suitable method. Suitable methods of analyzing the sample include microarray analysis, principle component analysis (PCA), and/or massive parallel RNA sequencing analysis (RNA-Seq).

The expression of the one or more hESC-signature genes in the sample can be compared with the expression of the one or more hESC-signature genes in a control. The control may be any suitable control. For example, the control can be airway basal cells obtained from the human at a previous time, airway basal cells obtained from one or more humans that do not have cancer, or airway basal cells obtained from one or more humans that do not smoke.

A different level of expression of the one or more hESC-signature genes in the sample compared to the level of expression of the one or more hESC-signature genes in the control is indicative of the presence cancer, the progression of cancer, or a predisposition to cancer in the human.

An increased or higher level of expression in the sample compared to the level of expression of the same hESC-signature genes in the control typically is a positive indication of the presence of cancer, a progression of cancer, or a predisposition to cancer in the human. The increased expression of the one or more hESC-signature genes as compared to the expression of the one or more hESC-signature genes in the control can be of any significant extent, e.g., 1.2-fold, 1.5-fold, 2-fold, 3-fold, 4-fold, 5-fold, 10-fold, 20-fold, 50-fold, 100-fold, 200-fold, or 500-fold higher expression. In a preferred embodiment, at least a 2-fold higher expression of the one or more hESC-signature genes in the sample as compared to the expression of the one or more hESC-signature genes in the control is a positive indication of the presence of cancer, a progression of cancer, or a predisposition to cancer in the human, especially when the control is airway basal cells obtained from the human at a previous time when the human was healthy (e.g., did not have a cancer, particularly a lung cancer), airway basal cells obtained from one or more humans that do not have cancer, or airway basal cells obtained from one or more humans that do not smoke.

A lack of expression or a similar or lower level of expression of the one or more hESC-signature genes in the sample as compared to the level of expression of the same hESC-signature genes in the control can be a negative indication of the presence of cancer, a progression of cancer, or a predisposition to cancer in the human. For example, when the control is airway basal cells obtained from the human at a previous time when the human was diagnosed with cancer, particularly a lung cancer, a lack of expression or a similar or lower level of expression of the one or more hESC-signature genes in the sample as compared to the level of expression of the same hESC-signature genes in the control can indicate the absence of cancer or the maintenance or regression of cancer.

The one or more hESC-signature genes can be any genes expressed by human embryonic stem cells, such as the genes disclosed by Assou et al. (Assou et al., Stem Cells, 25: 961-973 (2007)), including abhydrolase domain containing 9 (ABHD9) (EPHX3); barren homolog (Drosophila) (BRRN1) (NCAPH); cell division cycle 25A (CDC25A); CHK2 checkpoint homolog (S. pombe) (CHEK2); chromosome 14 open reading frame 115 (C14orf115); chromosome X open reading frame 15 (CXorf15); claudin 6 (CLDN6); cytochrome P450, family 26, subfamily A, polypeptide 1 (CYP26A1); defective in sister chromatid cohesion homolog 1 (S. cerevisiae) (DCC1) (DSCC1); deoxythymidylate kinase (thymidylate kinase) (DTYMK); DNA (cytosine-5-)-methyltransferase 3 alpha (DNMT3A); EPH receptor A1 (EPHA1); ets variant gene 4 (E1A enhancer binding protein, E1AF) (ETV4); FLJ20105 protein (FLJ20105) (ERCC6L); G protein-coupled receptor 19 (GPR19); G protein-coupled receptor 23 (GPR23) (LPAR4); gap junction protein, alpha 7, 45kDa (connexin 45) (GJA7) (GJC1); growth differentiation factor 3 (GDF3); helicase, lymphoid-specific (HELLS); homeo box (expressed in ES cells) 1 (HESX1); hypothetical protein FLJ10884 (ECAT11) (L1TD1); hypothetical protein MGC3101 (MGC3101) (DBNDD1); hypothetical protein PRO1853 (PRO1853) (C2orf56); interferon stimulated exonuclease gene 20 kDa-like 1 (ISG20L1) (AEN); KIAA0523 protein (KIAA0523) (WSCD1); lin-28 homolog (C. elegans) (LIN28); MCM10 minichromosome maintenance deficient 10 (S. cerevisiae) (MCM10); Nanog homeobox (NANOG); origin recognition complex, subunit 1-like (yeast) (ORC1L); origin recognition complex, subunit 2-like (yeast) (ORC2L); POU domain, class 5, transcription factor 1 (POU5F1); PR domain containing 14 (PRDM14); PWP2 periodic tryptophan protein homolog (yeast) (PWP2H); RNA binding motif protein 14 (RBM14); RNA, U3 small nucleolar interacting protein 2 (RNU3IP2) (RRP9); SLD5 homolog (SLD5) (GINS4); solute carrier family 5 (sodium-dependent vitamin transporter, member 6 (SLC5A6); teratocarcinoma-derived growth factor 1 (TDGF1); v-myb myeloblastosis viral oncogene homolog (avian)-like 2 (MYBL2); and zic family member 3 heterotaxy 1 (odd-paired homolog, Drosophila) (ZIC3).

A subset of hESC-signature genes is up-regulated in the basal cells of healthy smokers or in basal cells exposed to smoke or smoke extract in vitro and is referred to herein as the BC-S hESC-signature. In a preferred embodiment, the one or more hESC-signature genes are selected from the group of genes constituting the BC-S hESC-signature, i.e., the one or more hESC-signature genes are selected from the group consisting of BRRN1 (NCAPH); CDC25A; CHEK2; DCC1 (DSCC1); DTYMK; DNMT3A; EPHA1; FLJ20105 (ERCC6L); HELLS; MCM10; ORC1L; RBM14; RNU3IP2 (RRP9); SLD5 (GINS4); and MYBL2. In another embodiment, the one or more hESC-signature genes consist of the group of genes constituting the BC-S hESC-signature, i.e., consist of BRRN1 (NCAPH); CDC25A; CHEK2; DCC1 (DSCC1); DTYMK; DNMT3A; EPHA1; FLJ20105 (ERCC6L); HELLS; MCM10; ORC1L; RBM14; RNU3IP2 (RRP9); SLD5 (GINS4); and MYBL2.

Some hESC-genes are highly up-regulated in BC-S versus BC-NS. In a preferred embodiment, the one or more hESC-signature genes are selected from the group of genes consisting of BRRN1 (NCAPH); DCC1 (DSCC1); FLJ20105 (ERCC6L); MCM10; ORC1L; SLD5 (GINS4); and MYBL2. In another embodiment, the one or more hESC-signature genes consist of BRRN1 (NCAPH); DCC1 (DSCC1); FLJ20105 (ERCC6L); MCM10; ORC1L; SLD5 (GINS4); and MYBL2.

Some hESC-signature genes are up-regulated in BC-S versus BC-NS and are co-expressed in AdCa. In a preferred embodiment, the one or more hESC-genes are selected from the group consisting of BRRN (NCAPH), DCC1 (DSCC1), DTYMK, FLJ20105 (ERCC6L), MCM10, and MYBL2. In another embodiment, the one or more hESC-genes consist of BRRN (NCAPH), DCC1 (DSCC1), DTYMK, FLJ20105 (ERCC6L), MCM10, and MYBL2.

The inventive method can involve analyzing the sample to determine the expression of any number of hESC-signature genes, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 75, 100, or more hESC-signature genes, in any combination.

The tumor suppressor gene TP53—in addition to the one or more hESC-signature genes—can be evaluated in the sample for mutation and/or inactivation, which is further indicative of the presence of cancer, progression of cancer, and/or predisposition to cancer in the human. In particular, AdCa subjects with high expression of the BC-S hESC-signature exhibit higher frequency of mutations of the tumor suppressor gene TP53, suggesting that the initial acquisition of the TP53 inactivation molecular phenotype could be present in BC-S. TP53 is a tumor suppressor gene encoding phosphoprotein p53, which suppresses tumor formation by promoting apoptosis, activating cell cycle checkpoints, and inducing senescence (Yee et al., Carcinogenesis, 26: 1317-1322 (2005)).

The cancer can be any cancer. Typically, the cancer is lung cancer, such as adenocarcinoma, squamous cell carcinoma, large cell carcinoma, or small cell carcinoma. The cancer can have an aggressive clinical phenotype or a non-aggressive clinical phenotype.

The method can be utilized to detect cancer, a progression of cancer, or a predisposition to cancer in any human. In a preferred embodiment, the human is a smoker and/or has other risk factors for lung cancer.

The invention also provides an in vitro model for lung cancer, comprising airway basal cells that express one or more hESC-signature genes.

The expression of the one or more hESC-signature genes in the model is higher (e.g., 1.2-fold, 1.5-fold, 2-fold, 3-fold, 4-fold, 5-fold, 10-fold, 20-fold, 50-fold, 100-fold, 200-fold, or 500-fold higher) than expression of one or more hESC-signature genes in normal airway basal cells. In a preferred embodiment, the expression of the one or more hESC-signature genes in the model is at least 2-fold higher than the expression of the one or more hESC-signature genes in the normal airway basal cells. The expression of the one or more hESC-signature genes in the model can also be lower than expression of the one or more hESC-signature genes in normal airway basal cells.

The one or more hESC-signature genes can be any genes expressed by human embryonic stem cells, such as the genes disclosed by Assou et al. (Assou et al., Stem Cells, 25: 961-973 (2007)), including ABHD9 (EPHX3); BRRN1 (NCAPH); CDC25A; CHEK2; C14orf115; CXorf15; CLDN6; CYP26A1; DCC1 (DSCC1); DTYMK; DNMT3A; EPHA; ETV4; FLJ20105 (ERCC6L); GPR19; GPR23 (LPAR4); GJA7 (GJC1); GDF3; HELLS; HESX1; ECAT11 (L1TD1); MGC3101 (DBNDD1); PRO1853 (C2orf56); ISG20L1 (AEN); KIAA0523 (WSCD1); LIN28; MCM10; NANOG; ORC1L; ORC2L; POU5F1; PRDM14; PWP2H; RBM14; RNU3IP2 (RRP9); SLD5 (GINS4); SLC5A6; TDGF1; MYBL2; and ZIC3.

In a preferred embodiment, the one or more hESC-signature genes are selected from the group of genes constituting the BC-S hESC-signature, i.e., are selected from the group consisting of BRRN1 (NCAPH); CDC25A; CHEK2; DCC1 (DSCC1); DTYMK; DNMT3A; EPHA1; FLJ20105 (ERCC6L); HELLS; MCM10; ORC1L; RBM14; RNU3IP2 (RRP9); SLD5 (GINS4); and MYBL2. In another embodiment, the one or more hESC-signature genes consist of the group of genes constituting the BC-S hESC-signature, i.e., consist of BRRN1 (NCAPH); CDC25A; CHEK2; DCC1 (DSCC1); DTYMK; DNMT3A; EPHA1; FLJ20105 (ERCC6L); HELLS; MCM10; ORC1L; RBM14; RNU3IP2 (RRP9); SLD5 (GINS4); and MYBL2.

Some hESC-genes are highly up-regulated in BC-S versus BC-NS. In a preferred embodiment, the one or more hESC-signature genes are selected from the group of genes consisting of BRRN1 (NCAPH); DCC1 (DSCC1); FLJ20105 (ERCC6L); MCM10; ORC1L; SLD5 (GINS4); and MYBL2. In another embodiment, the one or more hESC-signature genes consist of BRRN1 (NCAPH); DCC1 (DSCC1); FLJ20105 (ERCC6L); MCM10; ORC1L; SLD5 (GINS4); and MYBL2.

Some hESC-signature genes are up-regulated in BC-S versus BC-NS and are co-expressed in AdCa. In a preferred embodiment, the one or more hESC-genes are selected from the group consisting of BRRN (NCAPH), DCC1 (DSCC1), DTYMK, FLJ20105 (ERCC6L), MCM10, and MYBL2. In another embodiment, the one or more hESC-genes consist of BRRN (NCAPH), DCC1 (DSCC1), DTYMK, FLJ20105 (ERCC6L), MCM10, and MYBL2.

The airway basal cells can express any number of hESC-signature genes, e.g., 1, 2, 3, 4, 5, 6, 7, 8, 9, 10, 11, 12, 13, 14, 15, 20, 25, 30, 40, 50, 100, 200, 500, or 1000 genes, in any combination.

The expression of the one or more hESC-signature genes in the in vitro model can be induced with smoke or smoke extract.

### EXAMPLES

The following examples further illustrate the invention but, of course, should not be construed as in any way limiting its scope.

In these examples, healthy nonsmokers (NS) are individuals in general good health, without a history of chronic lung disease, and without recurrent or recent acute pulmonary disease, and who do not have nicotine and/or cotinine in their urine. Healthy smokers (S) are individuals in general good health, without a history of chronic lung disease, and without recurrent or recent acute pulmonary disease, and who smoke any number of packs of cigarettes per year and have levels of nicotine and/or cotinine in their urine.

### Example 1

This example describes study populations and datasets.

Samples of LAE were obtained from 21 healthy nonsmokers and 31 healthy smokers. All individuals were evaluated at the Weill Cornell NIH Clinical and Translational Science Center and Department of Genetic Medicine Clinical Research Facility, under protocols approved by the Weill Cornell Medical College Institutional Review Board. Before enrollment, written informed consent was obtained from each individual.

Inclusion criteria for healthy nonsmokers comprised the following: males and females, at least 18 years old; provide informed consent; good health without history of chronic lung disease, including asthma, and without recurrent or recent (within 3 months) acute pulmonary disease; normal physical examination; normal routine laboratory evaluation, including general hematologic studies, general serologic/immunologic studies, general biochemical analyses, and urine analysis; HIV1 negative; al-antitrypsin level normal; normal PA and lateral chest X-ray; acceptable FVC—forced vital capacity, FEV1—forced expiratory volume in 1 sec, TLC—total lung capacity, and DLCO—diffusing capacity; normal electrocardiogram (sinus bradycardia and premature atrial contractions are permissible); not pregnant (females); no history of allergies to medications used in the bronchoscopy procedure; not taking any medications relevant to lung disease or having an effect on the airway epithelium; willingness to participate in the study; and self-reported nonsmokers, with smoking status validated by the absence of nicotine and cotinine in urine.

Exclusion criteria for healthy nonsmokers comprised the following: unable to meet the inclusion criteria; current active infection or acute illness of any kind; alcohol or drug abuse within the past 6 months; and evidence of malignancy within the past 5 years.

There were 21 healthy nonsmokers (15 male, 6 female; 42±9 yr; 9 African-American, 9 Caucasian, 4 other). All had normal lung function parameters. On the average, 6.8×10⁶ cells were recovered by bronchoscopy and brushing of the LAE, with >99% epithelium, 0.3±0.7% inflammatory cells. The LAE differential cell count included 55±4% ciliated cells, 12±4% secretory cells, 13±3% undifferentiated columnar cells, and 20±3% basal cells.

Inclusion criteria for healthy smokers comprised the following: males and females, at least 18 years old; provide informed consent; good health without history of chronic lung disease, including asthma, and without recurrent or recent (within 3 months) acute pulmonary disease; normal physical examination; normal routine laboratory evaluation, including general hematologic studies, general serologic/immunologic studies, general biochemical analyses, and urine analysis; HIV1 negative; α1-antitrypsin level normal; normal PA and lateral chest X-ray; acceptable FVC—forced vital capacity, FEV1—forced expiratory volume in 1 sec, TLC—total lung capacity, and DLCO—diffusing capacity; normal electrocardiogram (sinus bradycardia, premature atrial contractions are permissible); not pregnant (females); no history of allergies to medications used in the bronchoscopy procedure; not taking any medications relevant to lung disease or having an effect on the airway epithelium; willingness to participate in the study; and self-reported current daily smokers with any number of pack-yr, validated by urine nicotine 1000 ng/ml and cotinine>1000 ng/ml.

Exclusion criteria for healthy smokers comprised the following: unable to meet the inclusion criteria; current active infection or acute illness of any kind; alcohol or drug abuse within the past 6 months; and evidence of malignancy within the past 5 years.

There were 31 healthy smokers (21 male, 10 female; 44±7 yr; 19 African-American, 7 Caucasian, 5 other). All had normal lung function parameters. On the average, 6.4×10⁶ cells were recovered by bronchoscopy and brushing of the LAE, with >99% epithelium, 0.2±0.5% inflammatory cells. The LAE differential cell count included 49±9% ciliated cells, 11±4 secretory cells, 16±8% undifferentiated columnar cells, and 24±6% basal cells.

Samples of lung adenocarcinoma passaged in immunodeficient mice were derived from 4 individuals with primary lung adenocarcinoma (1 male, 3 female; 52±16 yr; 0 African Americans, 3 Caucasians, 1 other; 1 current smoker, 2 ex-smokers, and 1 smoking status unknown).

Samples of primary lung adenocarcinoma were collected at the time of resection from 193 patients with primary lung adenocarcinomas at Memorial Sloan-Kettering Cancer Center (MSKCC) as described by Chitale et al., Oncogene, 28: 2773-2783 (2009). The tissues were snap frozen in liquid nitrogen and stored at −80° C. By current WHO criteria, >90% of cases were classified as mixed subtype, based on combinations of areas of papillary, solid, acinar, and bronchioalveolar growth patterns. The tumor content (>70% tumor nuclei) was confirmed by frozen section, and the histopathologic diagnosis was verified by a pathologist independent of the investigators. The RNA extraction and microarray processing using the Affymetrix HG-U133A (91 samples) and HG-U133A 2.0 (102 samples) arrays have been described by Chitale et al. (Chitale et al., Oncogene, 28: 2773-2783 (2009)).

Previously published gene expression data from 193 of 199 primary lung AdCa of individuals undergoing surgery at Memorial Sloan-Kettering Cancer Center (MSKCC) was used for analysis (Chitale et al., Oncogene, 28: 2773-2783 (2009)). Independent publically available lung cancer datasets included data published by Landi et al. (AdCa, n=58) (Landi et al., PLoS One, 3: e1651 (2008)), Kuner et al. (AdCa, n=42; SCC, n=18) (Kuner et al., Lung Cancer, 63: 32-38 (2009)), Garber et al. (AdCa, n=40; SCC, n=13; SCLC, n=4; LCLC; n=4) (Garber et al., Proc. Natl. Acad. Sci. USA, 98: 13784-13789 (2001)), and Bild et al. (AdCa, n=58; SCC, n=53) (Bild et al., Nature, 439: 353-357 (2006)). The hESC datasets included data published by Avery et al. (n=3) (Avery et al., Stem Cells Dev., 17: 1195-1205 (2008)) and Denis et al. (GSE8590; n=2) (Denis et al., Stem Cells Dev., 20(8): 1395-1409 (2011)).

### Example 2

This example describes the collection of LAE.

Bronchoscopic brushings were used to obtain samples of large airway epithelium (LAE) from individuals via flexible bronchoscopy (Hackett et al., Am. J. Respir. Cell. Mol. Biol., 29: 331-343 (2003)). A 2.0 mm diameter brush was used to sample the epithelium of 3^{rd} and 4^{th} order bronchi, and cells were collected in 5 ml of ice cold bronchial epithelial basal medium (BEBM, Clonetics, Walkersville, Md.). An aliquot of 0.5 ml was used for differential cell count, and the remainder (4.5 ml) of the sample immediately was processed for RNA extraction. Total cell number was determined by counting on a hemocytometer. Differential cell count was assessed on sedimented cells prepared by centrifugation (CYTOSPIN™ 11, Shandon Instruments, Pittsburgh, Pa.) and stained with DIFFQUIK™ (Baxter Healthcare, Miami, Fla.). The LAE samples were free from stromal cellular elements and contained all major human airway epithelial cell subtypes including basal, ciliated, secretory and columnar cells, with basal cells contributing to ˜20% of the entire population.

### Example 3

This example describes the purification and culture of airway basal cells.

Based on the knowledge that basal cells, due to their unique pattern of integrin expression (Hicks et al., Exp. Cell Res., 237: 357-363 (1997)), exhibit superior capabilities of adhesion and migration and, as stem/progenitor cells, can self-renew and proliferate (Evans et al., Exp. Lung Res., 27: 401-415 (2001); Rock et al., Proc. Natl. Acad. Sci. USA, 106: 12771-12775 (2009)), as well as previous observations of the basal cell-like phenotype of airway epithelial cells grown in vitro (Araya et al., J. Clin. Invest., 117: 3551-3562 (2007)), a cell culture protocol was developed to obtain pure populations of basal cells from freshly isolated LAE samples.

Large airway epithelial cells were pelleted by centrifugation at 1250 rpm for 5 min and disaggregated from pellets with 0.05% trypsinethylenediaminetetraacetic acid (EDTA) for 5 min at 37° C., followed by the addition of Hank's Buffered Salt Solution (HBSS) with 15% fetal bovine serum (FBS). The cells were again pelleted (1250 rpm, 5 min) and then resuspended in 5 ml of bronchial epithelial basal medium (BEGM, Clonetics, Walkersville, Md.) and cultured at a density of 5×10⁵ in T25 plastic culture flasks (Becton Dickinson, Franklin Lakes, N.J.) in BEGM, supplemented with insulin, epidermal growth factor (0.5 ng/ml), hydrocortisone (0.5 mg/ml), transferrin (10 mg/ml), epinephrine (0.5 mg/ml), triiodothyronine (6.5 ng/ml), retinoic acid (0.1 ng/ml), and bovine pituitary extract (0.4% v/v) according to the manufacturer's instructions, but with substitution of the BEGM SINGLEQUOTS™ antibiotics with gentamycin (50 μg/ml; Sigma-Aldrich, St. Louis, Mo.), amphotericin B (1.25 μg/ml; GIBCO), and penicillinstreptomycin (50 μg/ml; GIBCO) (Karp et al., “Methods in molecular biology: epithelial cell culture protocols,” C. Wise, Ed. (Humana Press, Totowa), vol. 188, chapter 11 (2002)). Cultures were maintained in a humidified atmosphere of 5% CO₂ at 37° C. Culture medium was changed after 12 hr, and unattached cells were removed. Only a fraction of morphologically similar cells with a rounded shape and high nucleus-to-cytoplasm ratio were able to attach to the plastic surface, survive, and form multicellular clusters. The medium was changed every 2 days. At day 7 to 8 of culture when the cells were 70% confluent, the cells were removed from the plates with trypsin-EDTA. CYTOSPIN™ preparations were made to determine the percentage of basal cells using immunohistochemistry, and RNA was extracted.

### Example 4

This example describes the immunohistochemical characterization of basal cells.

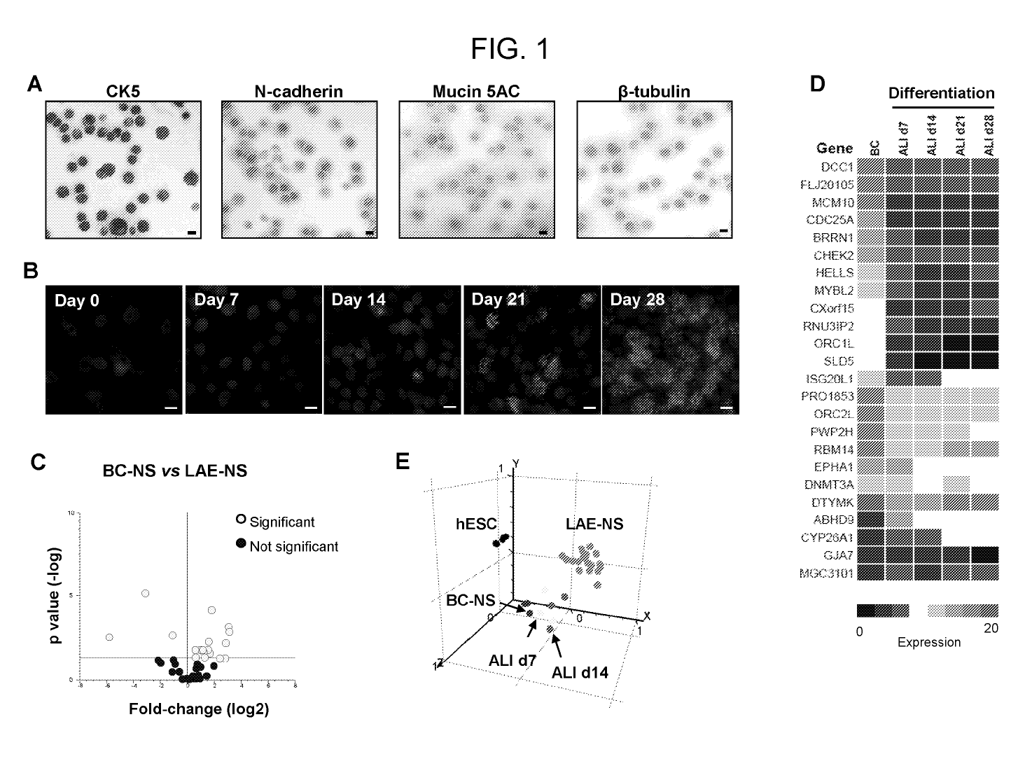

Purified basal cells were fixed in 4% paraformaldehyde for 15 min at 23° C. and then washed twice with 1× phosphate buffered saline (PBS). To enhance staining, an antigen recovery step was carried out by microwave treatment at 100° C., 15 min in citrate buffer solution (Labvision, Fremont, Calif.) followed by cooling at 23° C. for 20 min. Endogenous peroxidase activity was quenched using 0.3% H₂O₂, and normal serum matched secondary antibody was used for 20 min to reduce background staining Samples were incubated with the primary antibody overnight at 4° C., including rabbit anti-human cytokeratin 5 (K5) polyclonal antibody ( 1/50; Thermo Scientific) for confirmation of basal cell phenotype (Rock et al., Proc. Natl. Acad. Sci. USA, 106: 12771-12775 (2009) and Purkis et al., J. Cell Sci., 97 (Pt 1): 39-50 (1990)), mouse anti-human N-cadherin monoclonal antibody ( 1/2,500, Invitrogen) for exclusion of mesenchymal cells, mouse anti-human monoclonal mucin 5AC (MUC5AC) ( 1/50; Vector Laboratories, Burlingame, Calif.) for exclusion of secretory cells, and mouse anti-human β-tubulin IV monoclonal antibody (β4-tubulin) ( 1/2000 dilution; Biogenex, San Ramon, Calif.) for exclusion of ciliated cells, with isotype matched IgG (Jackson Immunoresearch Labs, West Grove, Pa.) as the negative control. The VECTASTAIN™ Elite ABC kit and AEC substrate kit (Dako, Carpinteria, Calif.) were used to visualize antibody binding. The cells were counterstained with hematoxylin and mounted using GVA mounting medium. Brightfield microscopy was done using a Nikon MICROPHOT™ microscope equipped with a Plan ×40 numerical aperture (NA) 0.70 objective lens. Images were captured with an Olympus DP70 CCD camera. This analysis demonstrated that the basal cultures were >95% positive for cytokeratin 5 (K5), a basal cell marker, and negative for mesenchymal cell marker N-cadherin, secretory cell marker mucin SAC, and ciliated cell marker β-tubulin IV (FIG. 1A).

### Example 5

This example describes the air-liquid model of airway epithelial cell differentiation.

The capacity of basal cells to generate differentiated progenies was assessed by culturing them using the air-liquid interface (ALI) model of airway epithelial differentiation (Karp et al., “Methods in molecular biology: epithelial cell culture protocols,” C. Wise, Ed. (Humana Press, Totowa), vol. 188, chapter 11 (2002), Rock et al., Proc. Natl. Acad. Sci. USA, 106: 12771-12775 (2009), and Hajj et al., Stem Cells, 25: 139-148 (2007)). After reaching 70 to 80% confluence, cells were trypsinized and seeded at a density of 2.0×10⁵ cells/cm² onto a 0.4 μm pore-sized COSTAR™ TRANSWELL™ inserts (Corning Incorporated, Corning, N.Y., via Fisher Scientific, Pittsburgh, Pa.) pre-coated with type IV collagen (Sigma). The initial culture medium consisted of a 1:1 mixture of DMEM and Ham's F-12 medium (GIBCO) containing 100 U/ml penicillin, 5% fetal bovine serum, 100 μg/ml streptomycin, 0.1% gentamycin, and 0.5% amphotericin. On the next day, the medium was changed to 1:1 DMEM/Ham's F12 with 2% ULTROSER™ G serum substitute (BioSerpa S.A., Cergy-Saint-Christophe, France). Cells were grown at 37° C., 5% CO₂, and the culture medium was changed every other day. Their apical surface was exposed to air as soon as they reached confluence, typically at culture day 1, to establish the ALI. Epithelial differentiation was assessed by monitoring transepithelial resistance (Rt) using MILLICELL-ERS™ epithelial ohmmeter (Millipore, Bedford, Mass.) and morphologically by determining of airway cilia formation, indicative for mucociliary epithelium. Cultures were considered differentiated if the Rt was more than 1000Ω/cm². To determine cilia formation, ALI cultures were washed once with 1×PBS and then fixed in 4% paraformaldehyde for 15 min at room temperature. After permeabilization with 0.2% Triton X-100 for 15 min at room temperature, the cells were incubated with mouse monoclonal anti-human β-tubulin IV ( 1/500 dilution; Biogenex, San Ramon, Calif.) for 1 hr at room temperature. Then, goat anti-mouse Cy3-conjugated AFFINIPURE™ (Jackson Immunoresearch, West Grove, Pa.) at 1/50 dilution was used as a secondary antibody. Nuclei were counter-stained with 4′,6-diamidino-2-phenylindole (DAPI, Invitrogen, Carlsbad, Calif.). Images were captured using an Olympus IX 70 fluorescence microscope with 60-fold magnification. Images were analyzed using METAMORPH™ software (Universal Imaging Corporation, Downingtown, Pa.). Pseudocolor images were formed by encoding Cy3 fluorescence in the red channel.

### Example 6

This example describes xenograft-based propagation of human lung adenocarcinomas.

Lung adenocarcinoma samples were obtained from 4 individuals for xenograft propagation in immunodeficient mice. Tumor tissue was mechanically dissociated with sterile scalpel blades and minced into approximately 1 mm in size. The tumor tissue was then enzymatically dissociated (using 10 mg/ml collagenase type IV (Sigma-Aldrich), and 4000U DNAase I (Sigma-Aldrich) for 1 hr, 37° C.) into single-cell suspensions. Cells of hematopoietic origin were depleted by magnetic bead separation using CD45 MICROBEADS™ (Miltenyi Biotec, Auburn, Calif.). Propagation of the human tumor cells was performed using a xenograft in vivo tumor model (Ito et al., Blood, 100: 3175-3182 (2002) and Wang et al., Blood, 104: 2893-2902 (2004)). Non-obese diabetic severe combined immunodeficiency (NOD.CB17-Prkdc^{scid}/J; NOD/SCID) interleukin 2 receptor (IL2R) gamma null immunocompromised mice (Jackson Laboratory; Bar Harbor, Me.) were maintained under specific pathogen-free conditions with a protocol approved at MSKCC. The CD45-negative cells were suspended in HBSS and MATRIGEL™ (BD Biosciences), (1:1 volume mixture) and then injected subcutaneously into the area of the mammary fat pad of 4 to 8 wk old mice with a 31-gauge insulin syringe (Becton Dickinson), and mice were monitored weekly for tumor growth. After 3 months, animals were sacrificed, and derived tumors were removed, dissociated to single cells and serially passaged at least twice in immunodeficient mice (102 cells/mouse), generating secondary tumors. After the final passage, tumor cells were processed for RNA isolation and gene expression analysis.

### Example 7

This example describes cDNA preparation, microarray processing, and data analysis.

Total RNA was extracted using a modified version of the TRIZOL™ method (Invitrogen, Carlsbad, Calif.), in which RNA is purified directly from the aqueous phase (RNEASY™ MINELUTE™ RNA purification kit, Qiagen, Valencia, Calif.), yielding 2 to 4 μg RNA per 10⁶ cells. RNA samples were stored in RNA SECURE™ (Ambion, Austin, Tex.) at −80° C. RNA integrity was determined by assessing an aliquot of each RNA sample on an Agilent Bioanalyzer (Agilent Technologies, Palo Alto, Calif.). A NANODROP™ ND-100 spectrophotometer (NanoDrop Technologies, Wilmington, Del.) was used to determine the concentration of RNA. Double stranded cDNA was synthesized from 1 to 2 μg of total RNA using the GENECHIP™ One-Cycle cDNA Synthesis Kit, followed by cleanup with GENECHIP™ Sample Cleanup Module, in vitro transcription reaction using the GENECHIP™ IVT Labeling Kit, and cleanup and quantification of the biotin-labeled cRNA yield by spectrophotometric analysis (all kits from Affymetrix, Santa Clara, Calif.).

All HG-U133 Plus 2.0 microarrays were processed according to Affymetrix protocols, hardware, and software, including being processed by the Affymetrix Fluidics Station 450 and Hybridization Oven 640 and scanned with an Affymetrix Gene Array Scanner 3000 7G. Overall microarray quality was verified by the following criteria: (1) RNA Integrity Number (RIN)≧7.0; (2) 375′ ratio for GAPDH≦3; and (3) scaling factor≦10.0 (Raman et al., BMC Genomics, 10: 493 (2009)).

The captured image data from the HG-U133 Plus 2.0 arrays was processed using MASS algorithm (Affymetrix Microarray Suite Version 5 software). MASS-processed data was normalized using GENESPRING™ version 7.3.1 (Agilent Technologies) by setting measurements <0.01 to 0.01, per array, by dividing the raw data by the 50^{th} percentile of all measurements, and, for identification of differentially expressed genes, additionally per gene, by dividing the raw data by the median expression level for all the genes across all arrays in a dataset.

Criteria for differentially expressed genes were: (1) P call of “Present” in ≧20% of samples for study groups including more than 20 samples (i.e., LAE of healthy nonsmokers and LAE of healthy smokers) or in at least 2 of 4 samples (for basal cells of healthy nonsmokers, basal cells of healthy smokers, human lung adenocarcinoma cells passaged in mice); the gene was considered expressed in a particular study group if it met this P call criteria; and (2) p<0.05 using a t test with a Benjamini-Hochberg correction to limit the false positive rate. In selected experiments involving small-size study groups (n=4), comparisons were performed both with and without applying the Benjamini-Hochberg correction, to increase the sensitivity of analysis. However, in all cases, confirmatory comparisons with Benjamin-Hochberg correction were performed. Forty hESC-specific genes were selected for the analysis based on the meta-analysis of the hESC transcriptome (Assou et al., Stem Cells, 25: 961-973 (2007)).

To provide a cumulative measure of an individual signature expression in AdCa samples, signature-specific indexes were calculated for each individual AdCa sample as a number of signature genes with the expression level above the median in AdCa subjects.

To ensure that differential expression of hESC-specific genes in basal cells of smokers was not due to global nonspecific transcriptome modification, expression of a set of 10 well-defined housekeeping genes (Eisenberg et al., Trends Genet., 19: 362-365 (2003)) was comparatively analyzed in basal cells of healthy smokers and those of nonsmokers. The list of housekeeping genes analyzed included: actin, beta (ACTB), Rho GDP dissociation inhibitor (GDI) alpha (ARHGDIA), ATPase, H+ transporting, lysosomal 13 kDa, V1 subunit G isoform 1 (ATP6V1G1), endosulfine alpha (ENSA), glyceraldehyde-3-phosphate dehydrogenase (GAPDH), lactate dehydrogenase A (LDHA), ribosomal protein S18 (RPS18), ribosomal protein L19 (RPL19), ribosomal protein S27a (RPS27A), and ribosomal protein L32 (RPL32). Unsupervised hierarchical clustering of study samples described above was carried out in GENESPRING™ software based on expression of detected hESC-specific genes using the MASS-analyzed data with the standard and correlation as similarity measure and the complete linkage clustering algorithm. The results were validated by using alternative clustering settings with Pearson correlation as similarity measure and the average linkage clustering algorithm.

To visualize the contribution of variability in hESC-specific gene expression to transcriptome differences between various groups, principal component analysis (PCA) was performed using all hESC-specific genes present in at least 20% of samples (for study groups including more than 20 samples as with the samples of LAE of healthy nonsmokers and smokers), and in at least 2 of 4 of samples (for BC of healthy nonsmokers, and smokers, and lung adenocarcinoma cells). These analyses were carried out using GENESPRING™ by mean centering and scaling of microarray normalized intensity values of all subjects or the average for each of the three groups in order to assign the general variability in the data to a reduced set of principal components (Jolliffe, “Principal component analysis,” Spinger-Verlag, N.Y., ed. 2 (2002)). The first 3 principal components containing most of the variance-based information were visualized in 3-dimensional space.

To evaluate the relative contribution of the differences in hESC-specific gene expression to the global transcriptional differences between basal cells of healthy smokers and basal cells of healthy nonsmokers, the variability (as measured by the first 3 principal components) determined by PCA on hESC-specific gene probe sets was compared to that revealed by PCA of the same study groups using all gene probe sets detected in at least 2 of 4 samples.

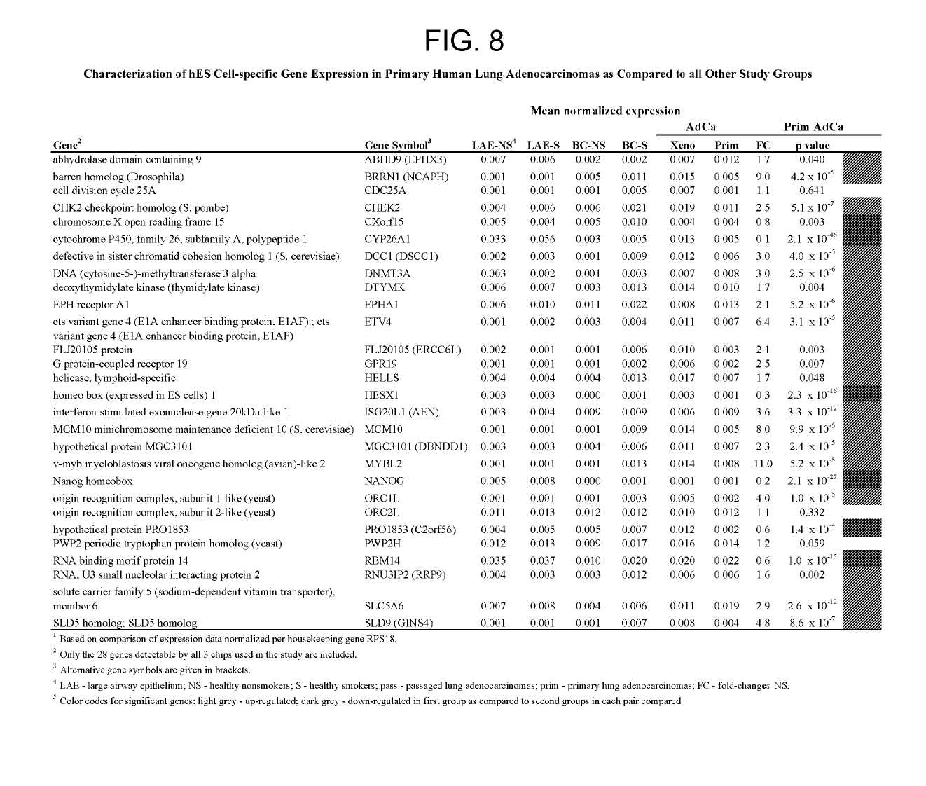

For identification of hESC-specific genes differentially expressed in primary lung adenocarcinoma samples versus LAE of healthy nonsmokers, multiplatform expression data was normalized per housekeeping gene RPS18 (Eisenberg et al., Trends Genet., 19: 362-365 (2003)) that was previously described as a stable reference gene for carcinoma gene expression studies (Lallemant et al., BMC Mol. Biol., 10: 78 (2009)) and exhibited a very high correlation (Pearson's correlation coefficient>0.9, p<0.05) with 11 out of 15 (73%) recently identified top stable housekeeping genes including RPS13, RPL27, RPS20, RPL13A, RPL9, RPL24, RPL22, RPS29, RPS16, RPL4, and RPL6 (de Jonge et al., PLoS One, 2: e898 (2007)) across the analyzed dataset (data not shown). This analysis was restricted to 28 hESC-specific genes (ABHD9 (EPHX3); BRRN1 (NCAPH); CDC25A; CHEK2; CXorf15; CYP26A1; DCCT (DSCC1); DNMT3A; DTYMK; EPHA; ETV4; FLJ20105 (ERCC6L); GPR19; HELLS; HESX1; ISG20L1 (AEN); MCM10; MGC3101 (DBNDD1); MYBL2; NANOG; ORC1L; ORC2L; PRO1853 (C2orf56); PWP2H; RBM14; RNU3IP2 (RRP9); SLC5A6; and SLD5 (GINS4)) whose expression can be analyzed by all three microarray platforms used, with the expression data set forth in FIG. 8.

The raw data are publically available at the Gene Expression Omnibus (GEO) website (GSE19722). Independent lung cancer datasets were analyzed using ONCOMINE database (Rhodes et al., Neoplasia, 6: 1-6 (2004)) or using GENESPRING™ software (for databases imported from the GEO).

NCI-H522, NCI-HI299, NCI-H338, and A549 lung carcinoma cell lines were purchased from ATCC (Rockville, Md.) and cultured according to the ATCC protocols. Expression of selected hESC genes was analyzed using specific TAQMAN™ assays (Applied Biosystems, Foster City, Calif.) as described (Shaykhiev et al., Cell. Mol. Life Sci., 68(5): 877-892 (2011)).

Kaplan-Meier survival analysis was carried out using MedCalc version 11.3.3. Difference in survival between the groups was analyzed with the log-rank test. Clinical characteristics were compared using Chi-square test (for categorical variables) and Kolmogorov-Smimov test (for continuous variables).

### Example 8

This example describes massive parallel mRNA sequencing.

The transcriptome of the basal cells of healthy nonsmokers (BC-NS) and basal cells of healthy smokers (BC-S) was additionally studied using massive parallel RNA sequencing (RNA-Seq). For RNA that met the same quality criteria as for microarray, 6 μg of total RNA per subject was processed according to Illumina's mRNA Sequencing Sample Preparation Guide #1004898 Rev D. The mRNA was purified and isolated from the total RNA with poly-A selection and fragmented, cDNA was synthesized, and adaptors ligated to both ends. The product was purified and enriched with PCR to create the final cDNA library. The cDNA library then was bound to the flow cell by hybridizing the fragments to single-stranded, adapter-ligated fragments bound to the flow cell surface. Bridge amplification then was performed to create millions of dense clusters using the Illumina Cluster Station. The clusters were sequenced with a sequencing primer by incorporation of fluorescent nucleotides (one base/cycle) for 43 cycles on the Illumina Genome Analyzer II according to Illumina's Single-Read Sequencing User Guide GAII 1004831 Rev A protocol. After each cycle, each tile of the flow cell was imaged for each nucleotide. This cycle was repeated, one base at a time, generating a series of images each representing a single base extension at a specific cluster.

Image analysis, base calling and read quality filtering were performed by the Solexa analysis software that export FASTQ formatted sequence files for each subject. The Bowtie alignment algorithm v0.11.3 was used in Partek Genomics Suite v6.5 with the UCSC hg18 reference sequence to map between 6.49 to 16.49 million reads for each samples. These 42 by reads mapped to over 32,000 transcripts genome-wide and generated gene expression levels for 20,713 unique genes. Partek estimates the maximum likelihood of each isoform being expressed using an expectation/maximization algorithm to calculate the raw counts and then normalizes to get the reads per kilobase of exon model per million mapped reads (RPKM). In the RNA-Seq-based analysis, expression of all 40 hESC-specific genes was assessed in BC-NS and BC-S (n=2 in each group). The gene was considered expressed in basal cells if RPKM>0 in at least 2 of 4 samples.

Criteria for up-regulated genes were detectable expression in at least 50% of samples with at least 2-fold increased average expression in BC-S versus BC-NS and at least 1.5-fold increased expression level in each BC-S sample as compared to the BC-NS sample with a highest expression for a given hESC-specific gene. Criteria for down-regulated genes were detectable expression in at least 50% of samples with at least 2-fold decreased average expression in BC-S versus BC-NS and at least 1.5-fold decreased expression level in each BC-S sample as compared to the BC-NS sample with a lowest expression for a given hESC-specific gene.

### Example 9

This example demonstrates that hESC-signature genes are expressed in adult human airway epithelium.

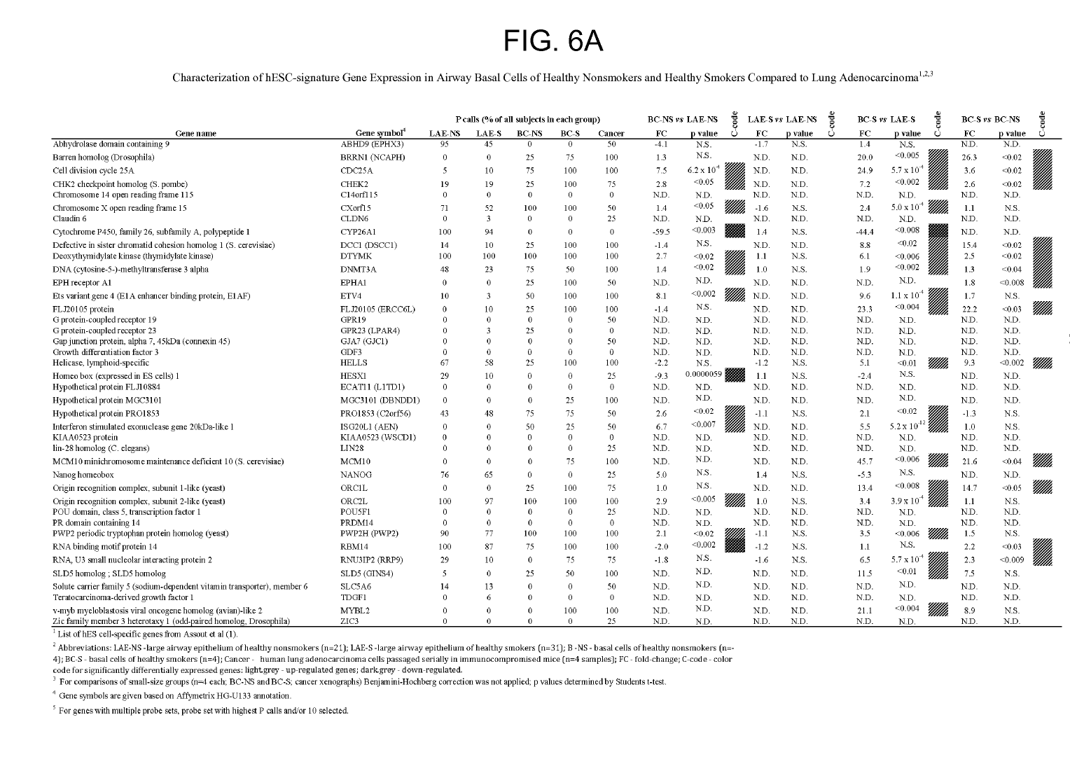

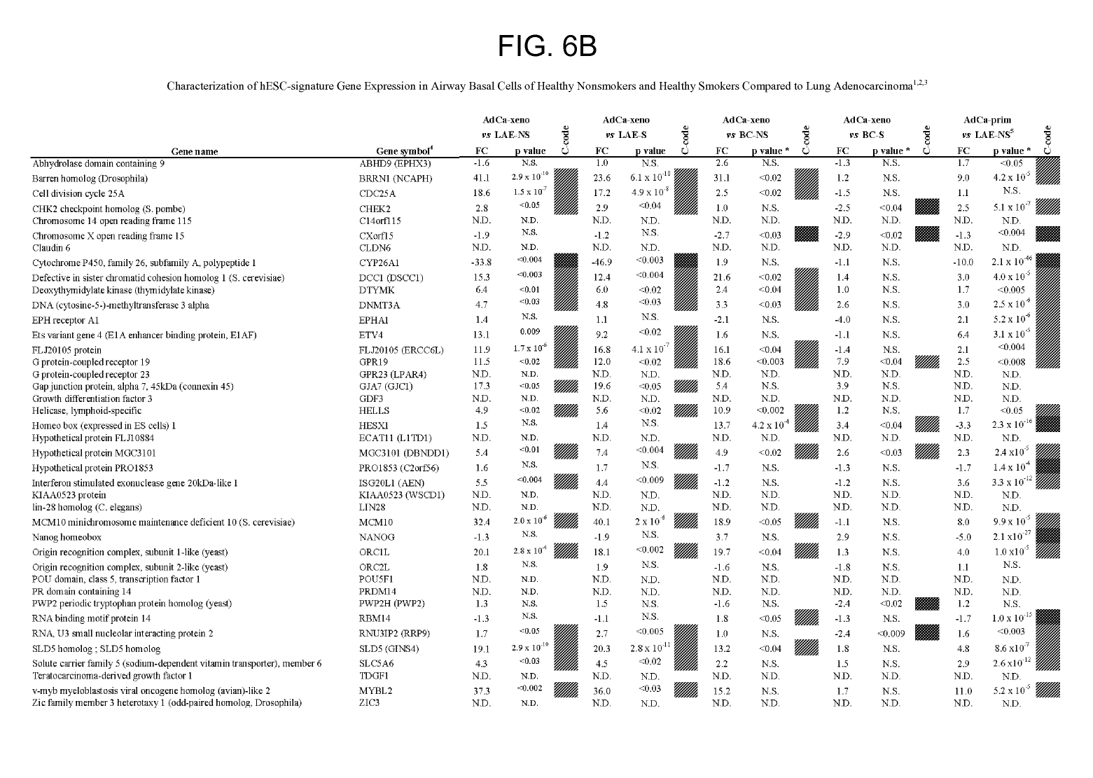

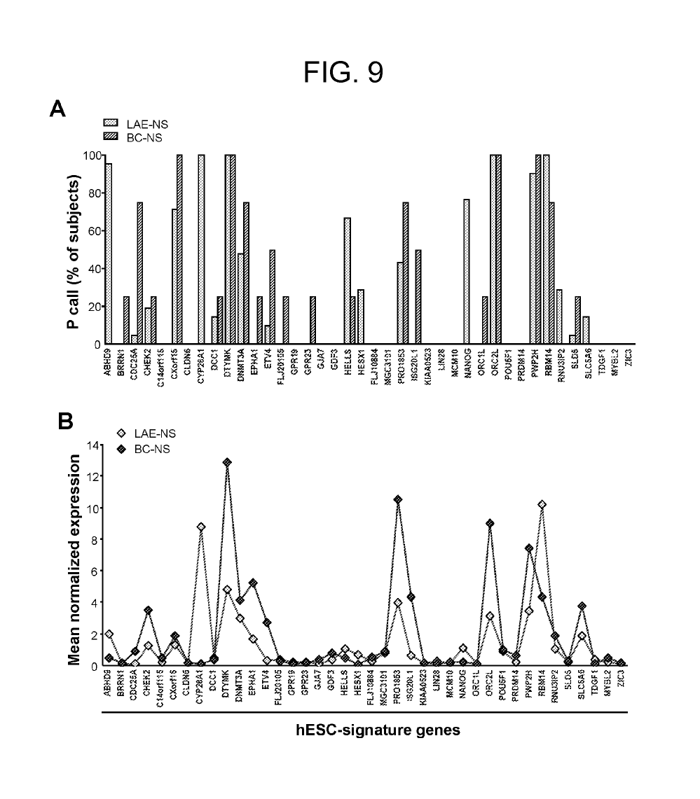

The LAE and LAE-derived basal cells of healthy nonsmokers (LAE-NS and BC-NS, respectively) were analyzed for expression of the 40 hESC-signature genes. Remarkably, 25% of hESC-signature genes were detected in at least 50% of samples in the both groups, and 10% were detected in all samples analyzed (FIG. 9A; FIG. 6). However, the expression pattern of these genes in the LAE compared to the basal cells was not identical. Some were expressed in the LAE, but not in the basal cells (e.g., ABHD9, CYP26A1, HESX1, and NANOG), i.e., were cell differentiation-associated. Others (e.g., CDC25A, DTYMK, EPHAl, ISG20L1, and ORC2L) were expressed more abundantly in the basal cell population (FIG. 9B).

Among 27 hESC-signature genes detected in either LAE-NS or BC-NS, 15 were differentially expressed between these 2 groups, with the majority (12 of 15) significantly up-regulated in basal cells (FIG. 1C). Microarray analysis of basal cell differentiation in vitro in ALI revealed that while expression of a minor subset of hESC-signature genes increased with cell differentiation, including ABHD9 and CYP26A1, similar to the in vivo data from the LAE-NS, the majority of hESC genes, including DCC1, FLJ20105, MCM10, CDC25A, BRRN1, CHEK2, HELLS, MYBL2, CXorf15, RNU3IPU, ORC1L, SLD5, ISG201L, PRO1853, ORC2L, PWP2H, RBM14, EPHAl, DNMT3A, DTYMK, and GJA7, are down-regulated during airway epithelial differentiation (FIG. 1D). The major changes occurred within the first 2 weeks of the differentiation process (FIG. 1D). Consistently, principal component analysis (PCA) revealed a significant difference between LAE and basal cells based on the expression of hESC-signature genes, with basal cells clustered closer to hESCs but shifted toward completely differentiated in vivo LAE during the first 2 weeks of differentiation in ALI (FIG. 1E). Thus, adult human airway epithelium, in its normal healthy state, maintains some elements of the hESC-like molecular program, with relative overall enrichment in the basal cell population.

### Example 10

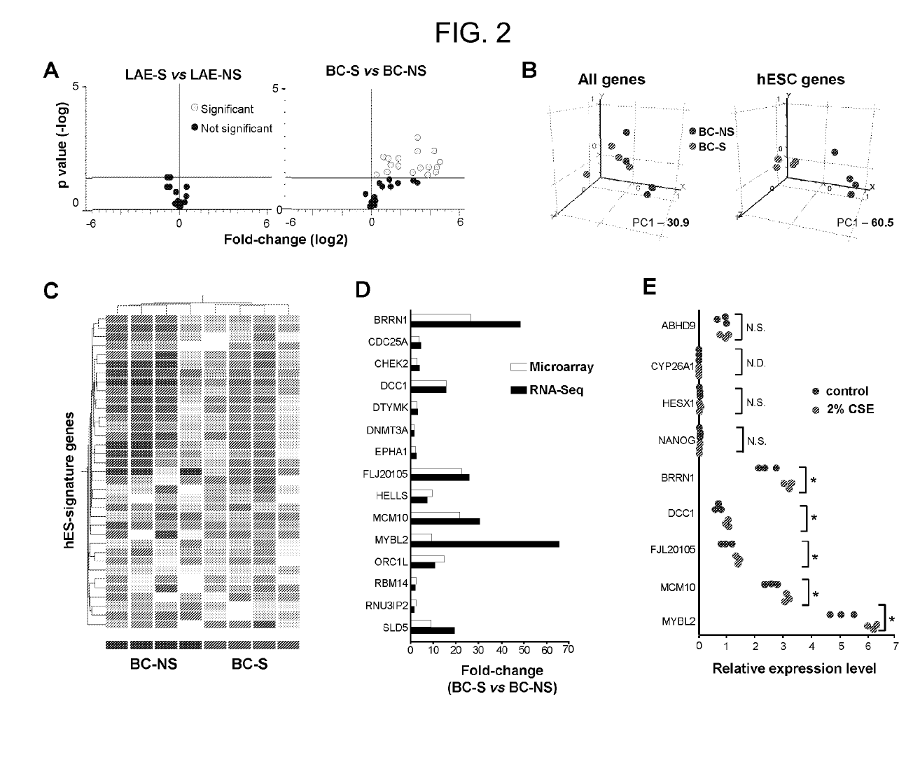

This example demonstrates that smoking activates a hESC-signature in airway basal cells.

Whereas the expression of hESC-signature genes by the LAE of healthy smokers (LAE-S) did not differ significantly from that of healthy nonsmokers (FIG. 2A, left panel), basal cells of healthy smokers (BC-S) exhibited a broad up-regulation of hESC-signature genes (FIG. 2A; right panel). Of the 35 hESC-signature gene probe sets expressed in BC-NS and/or BC-S, 18 (51%) probe sets corresponding to 13 (33%) hESC-signature genes (BRRN1 (NCAPH); CDC25A; CHEK2; DCC1 (DSCC1); DTYMK; DNMT3A; EPHA1; FLJ20105 (ERCC6L); HELLS; MCM10; ORC1L; RBM14; and RNU3IP2 (RRP9)) were differentially expressed between these 2 groups, with all significantly up-regulated in BC-S (FIG. 6). Notably, 10 of these 13 genes (77%) were not detected in BC-NS indicative of their de novo expression in BC-S (FIG. 6).

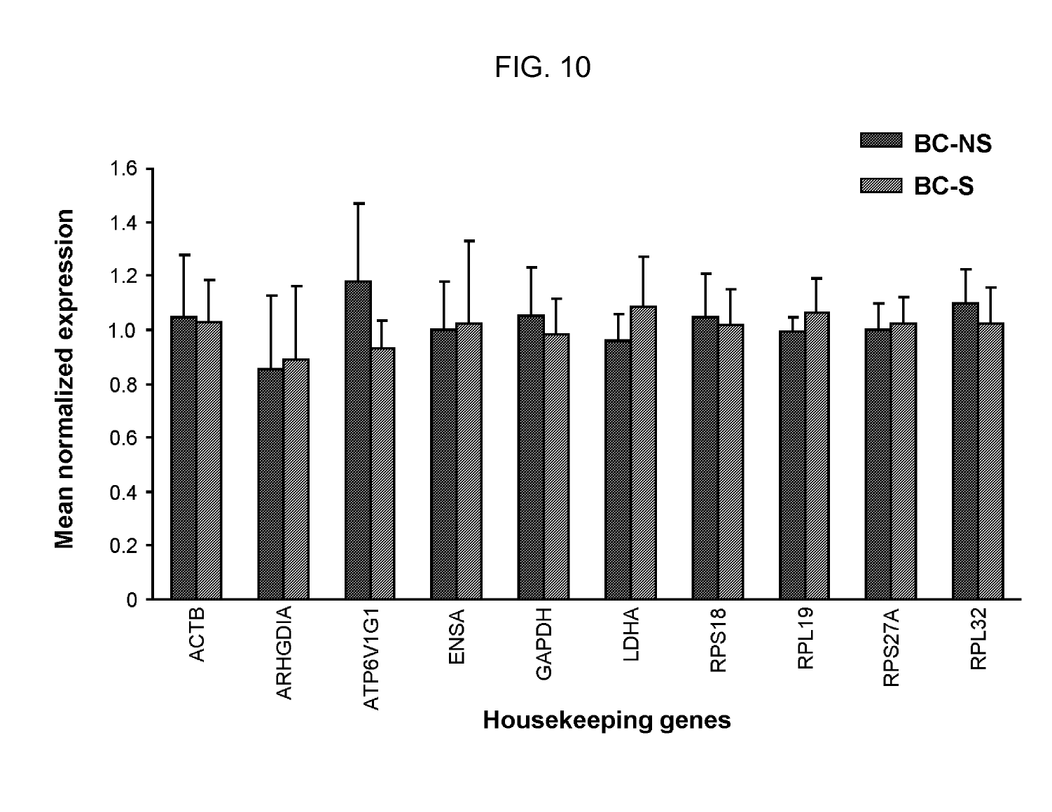

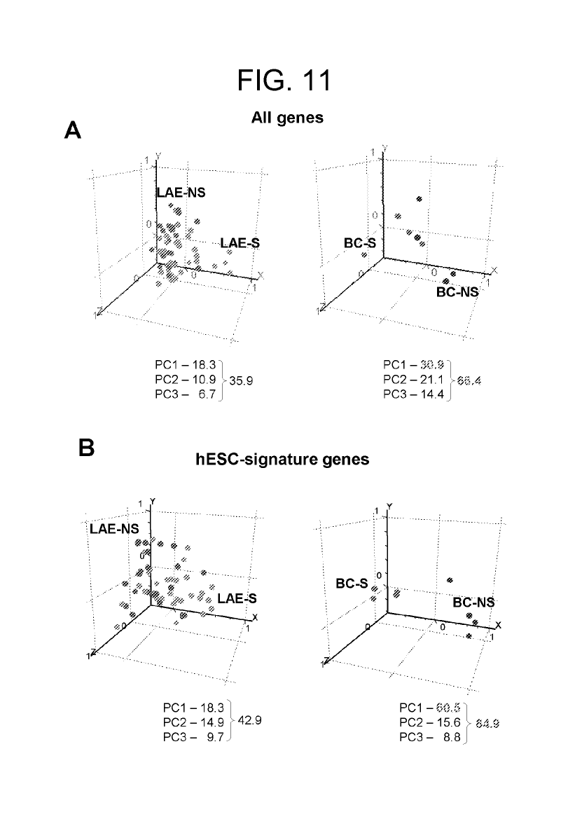

These differences were not due to the nonspecific global basal cell transcriptome activation by smoking, as expression of housekeeping genes (ACTB, ARGHGDIA, ATP6V1G1, ENSA, GAPDH, LDHA, RPS18, RPL19, RPS27A, and RPL32) was unchanged (FIG. 10). Moreover, PCA analysis revealed that, whereas the effect of cigarette smoking on the entire transcriptome had only limited contribution to variability between the different groups for both LAE (FIG. 11A; left panel) and basal cells (FIG. 2B, left panel), healthy smokers and healthy nonsmokers were completely segregated from each other based on the expression of hESC-signature genes in their basal cells (FIG. 2B, right panel), but not in the LAE (FIG. 11B, left panel). Remarkably, in the PCA-based comparison of BC-S and BC-NS, the first principal component, which accounts for most of the variability in the data (Jolliffe, “Principal component analysis,” Spinger-Verlag, N.Y. (2002)), increased from 30.9% in the transcriptome-wide analysis to 60.5% in the hESC-signature gene-restricted analysis (FIG. 2B), while it remained unchanged for the LAE (FIG. 11). Consistent with the PCA, unsupervised hierarchical cluster analysis genes completely separated BC-S from BC-NS based on the expression of hESC-signature (FIG. 2C).

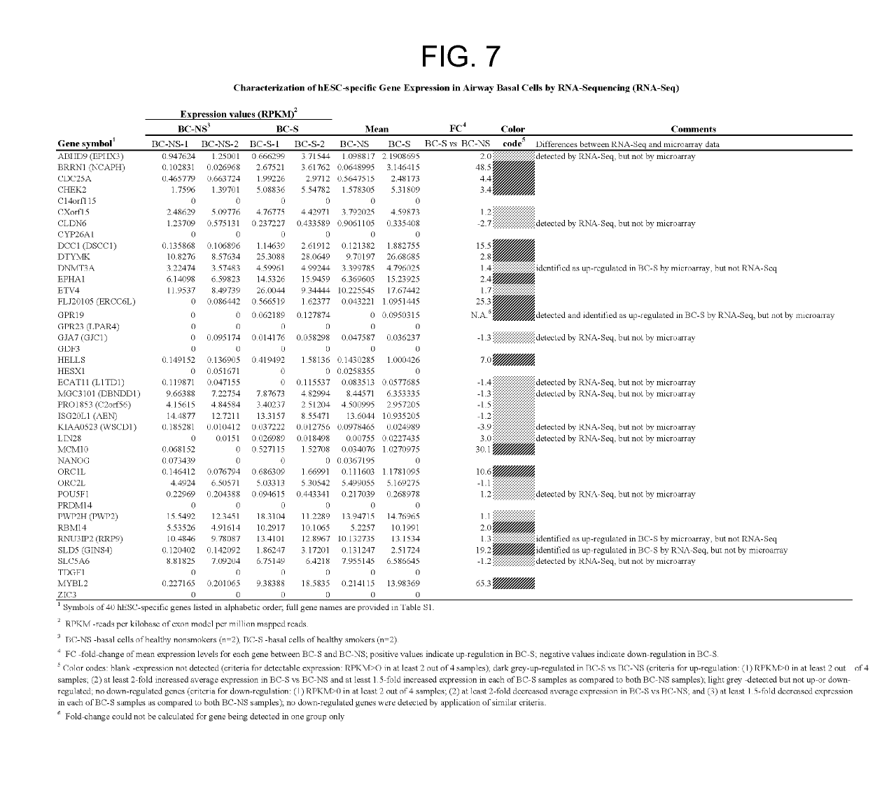

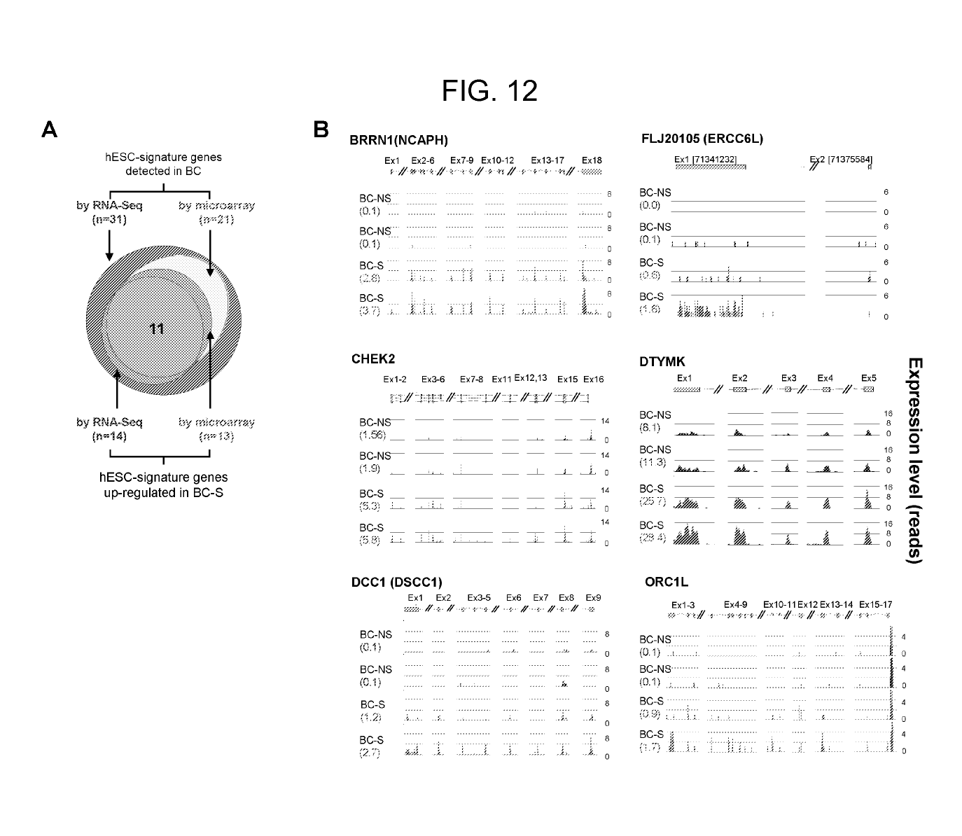

Massive parallel RNA sequencing (RNA-Seq) has recently emerged as highly sensitive and replicable technology for measuring mRNA expression, detecting novel transcripts and identifying differentially expressed genes, especially those with relatively low expression (Marioni et al., Genome Res., 18: 1509-1517 (2008)), as in the case with the low-abundant hESC-signature genes in adult tissues. RNA-Seq was used to validate differential expression of hESC-signature genes in BC-S versus BC-NS. This analysis revealed overlap between differentially expressed hESC-signature genes identified by RNA-Seq and microarray (FIG. 12A). Consistently, all 13 hESC-signature genes identified by microarray as up-regulated in BC-S (BRRN1 (NCAPH); CDC25A; CHEK2; DCC1 (DSCC1); DTYMK; DNMT3A; EPHA1; FLJ20105 (ERCC6L); HELLS; MCM10; ORC1L; RBM14; and RNU3IP2 (RRP9)) displayed similar direction of expression differences in the RNA-Seq analysis (FIG. 2D, FIG. 12B). RNA-Seq revealed 2 additional hESC-signature genes (SLD5 (GINS4) and MYBL2) up-regulated in BC-S (FIG. 7). Thus, using both methods, a total of 15 hESC-signature genes were found up-regulated in BC-S compared to BC-NS (BRRN1 (NCAPH); CDC25A; CHEK2; DCC1 (DSCC1); DTYMK; DNMT3A; EPHA1; FLJ20105 (ERCC6L); HELLS; MCM10; ORC1L; RBM14; RNU3IP2 (RRP9); SLD5 (GINS4); and MYBL2). This set of genes constitutes the BC-S hESC-signature.

To determine whether up-regulation of the hESC-signature genes in BC-S was a result of the direct effect of cigarette smoke on basal cells, BC-NS were stimulated in vitro with 2% cigarette smoke extract (CSE), which is a non-toxic concentration for airway epithelial cells (Shaykhiev et al., Cell. Mol. Life Sci., 68(5): 877-892 (2011)). Indeed, 2% CSE significantly up-regulated expression of the hESC-signature genes found induced in BC-S in vivo, but not those whose expression was unchanged in BC-S in vivo and associated with airway epithelial differentiation in vivo and/or in vitro (FIG. 2E).

Accordingly, smoking activates a hESC signature (BC-S hESC-signature) in airway basal cells but not in LAE.

The reason for this apparent discrepancy may relate to the fact that basal cells represent only ˜20-25% of the LAE, while samples of cultured basal cells are >95% pure. Smoking is known to induce contrasting effects on different cell populations of the airway epithelium. For example, there is loss and functional defects of ciliated cells, the predominant airway epithelial cell type, opposed to the increased proliferation of basal cells contributing to ciliated cell replenishment in the airway epithelium of smokers (Jeffery et al., Adv. Exp. Med. Biol., 144: 399-409 (1982)).

Cigarette smoking is the dominant environmental carcinogenic stressor for airway epithelial cells, including basal cells which constitute the stem/progenitor cell pool of the airway epithelium and are capable of self-renewing and differentiating into specialized cellular elements (Hajj et al., Stem Cells, 25: 139-148 (2007); Hong et al., Nature, 460: 1132-1135 (2009); Inayama et al., Am. J. Pathol., 134: 539-549 (1989); Rock et al., Proc. Natl. Acad. Sci. USA, 106: 12771-12775 (2009)). Cigarette smoking is capable of evoking dramatic changes in the epithelial gene expression program (Harvey et al., i J. Mol. Med., 85: 39-53 (2007); Spira et al., Nat. Med., 13: 361-366 (2007)) and inducing oncogenic mutations and epigenetic modifications relevant to lung cancer (Sato et al., J Thorac. Oncol., 2: 327-343 (2007); Wistuba et al., Oncogene, 21: 7298-7306 (2002)). In susceptible individuals, cigarette smoking is responsible for inducing airway epithelial cells to change their normal differentiation pattern, undergo increased proliferation and eventually become malignant. Basal cell hyperplasia and squamous metaplasia are the earliest airway epithelial lesions associated with smoking-induced carcinogenesis (Auerbach et al., N. Engl. J. Med., 256: 97-104 (1957); Wistuba et al., Oncogene, 21: 7298-7306 (2002); Wistuba et al., Ann. Rev Pathol., 1: 331-348 (2006)). It is possible that smoking-associated oxidative stress is responsible for selective activation of the hESC-related program in the airway basal cell population. Consistent with this idea, resistance to oxidative stress is a feature of stem cells (Diehn et al., Nature, 458: 780-783 (2009)), thereby raising a possibility that in response to smoking-induced oxidative stress, airway basal cells, by contrast to differentiated cells, instead of being damaged, enrich their sternness-related hESC-like program as a compensatory mechanism necessary for tissue repair.

Basal cells, located below the layer of differentiated and columnar cells, appear to sense cigarette smoke, possibly because the intercellular junctional barrier of the lung epithelium is compromised by cigarette smoking (Boucher et al., Lab. Invest., 43: 94-100 (1980); Shaykhiev et al., Cell. Mol. Life Sci., 68(5): 877-892 (2011)), thereby making the basal cell compartment accessible to components of cigarette smoke. In addition, basal cells can directly sample luminal content by extending their processes across the epithelial layer (Shum et al., Cell, 135: 1108-1117 (2008)). Indeed, direct exposure of basal cells from healthy nonsmokers to cigarette smoke extract in vitro has been demonstrated to result in the acquisition of the hESC-signature similar to that induced in BC-S in vivo.

Interestingly, the cultured basal cells maintain their altered hESC-like gene expression. Since the basal cells were proliferated in culture over 7 days, it is likely that stable changes to the basal cell genome and/or epigenome induced by smoking in vivo allowed them to maintain their phenotype after they have been removed from the in vivo microenvironment. The ability of smoking to cause mutations and epigenetic modifications in the airway epithelium is well documented (Sato et al., J Thorac. Oncol., 2: 327-343 (2007); Wistuba et al., Oncogene, 21: 7298-7306 (2002)). The overall hESC-signature gene expression markedly decreased following basal cell differentiation into the ciliated epithelium in vitro, thereby suggesting that the regulatory mechanisms controlling the expression of these genes in vivo also were largely preserved in vitro, and the observed increased hESC-signature gene expression in BC-S versus BC-NS was due to in vivo smoking-induced reprogramming.

### Example 11

This example demonstrates that a smoking-induced basal cell hESC-signature contributes to the hESC-like phenotype of human lung adenocarcinoma.

Based on previous observations that a subset of lung adenocarcinomas (AdCa) exhibit a hESC-like molecular profile (Hassan et al., Clin. Cancer Res., 15(20): 6386-6390 (2009)), commonality in the pattern of hESC-signature genes overexpressed in BC-S (BC-S hESC-signature) and this type of lung cancer was investigated.

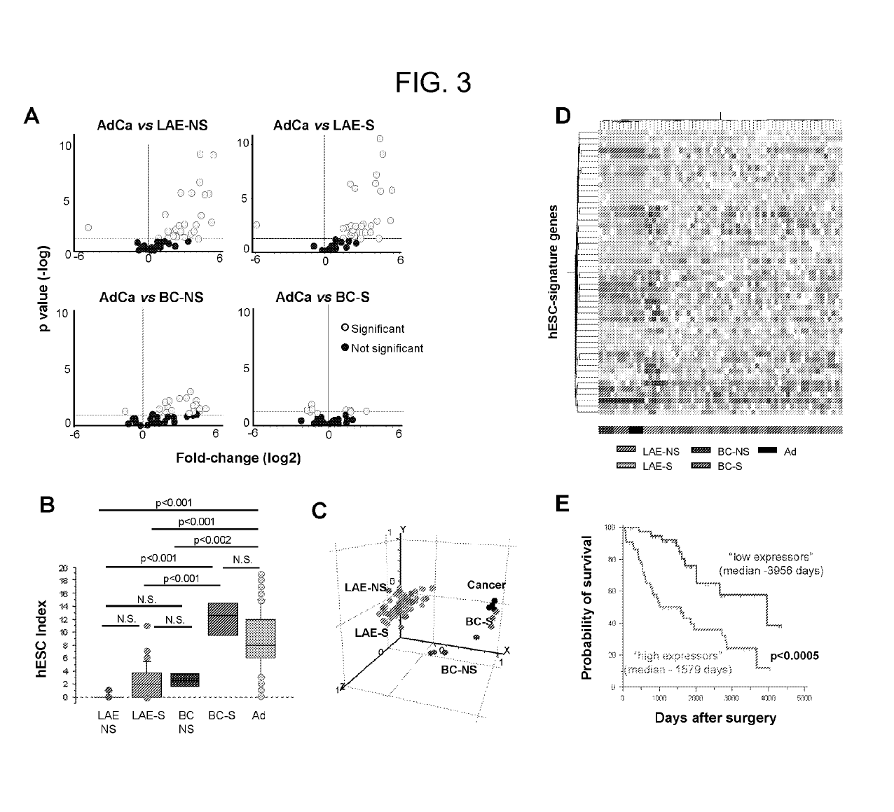

First, the hESC-signature expression in primary human lung AdCa cells that had been passaged serially in NOD/SCID/IL2Rgamma-null (Ito et al., Blood, 100: 3175-3182 (2002)) immunodeficient mice was assessed. This approach permitted evaluation of a pure epithelial compartment of carcinoma cells without the complicating contamination of non-cancer cellular elements contributing to tumor microenvironment (Frese et al., Nat. Rev. Cancer, 7: 645-658 (2007)) that might exhibit hESC-like molecular features (Cesselli et al., Circ. Res., 104: 1225-1234 (2009); Howell et al., Ann. N.Y. Acad. Sci., 996: 158-173 (2003)). Since the HG-U133 Plus 2.0 array is human gene-specific, any contribution of the murine cellular elements to the analysis results was circumvented. The analysis revealed that 20 of 40 hESC-signature genes were significantly up-regulated in AdCa xenografts as compared to LAE-NS (FIG. 3A, left upper panel) and LAE-S (FIG. 3A, right upper panel). Strikingly, however, whereas the AdCa-xenografts displayed a considerable number of up-regulated hESC-signature genes compared to BC-NS (FIG. 3A, left lower panel), the hESC-signature induced in BC-S was remarkably similar to that of AdCa cells (FIG. 3A, right lower panel). Consistently, comparative analysis of the hESC index, i.e., a cumulative measure of overexpression of hESC-signature genes (calculated as an average number of hESC-signature genes having expression level above the median level in the LAE-NS), revealed significant increased average expression of hESC-signature genes in AdCa versus BC-NS (p<0.002), whereas there was no significant difference between AdCa and BC-S (FIG. 3B). Of 15 BC-S hESC-signature genes, 12 (80%) were among those 19 overexpressed in AdCa cells (FIG. 6). Both PCA (FIG. 3C) and unsupervised hierarchical clustering (FIG. 3D) demonstrated that, based on the expression of hESC-signature genes, BC-S were completely segregated from the LAE and BC-NS, and exhibited a distribution pattern similar to AdCa cells.

Next, the hESC-signature gene expression was assessed in primary tumor tissues obtained from 193 patients with lung AdCa (Chitale et al., Oncogene, 28: 2773-2783 (2009)). Consistent with the xenograft data, 19 of 28 (ABHD9 (EPHX3); BRRN1 (NCAPH); CHEK2; DCC1 (DSCC1); DTYMK; DNMT3A; EPHA; ETV4; FLJ20105 (ERCC6L); GPR19; HELLS; MGC3101 (DBNDD1); ISG20L1 (AEN); MCM10; ORC1L; RNU3IP2 (RRP9); SLD5 (GINS4); SLC5A6; and MYBL2 out of ABHD9 (EPHX3); BRRN1 (NCAPH); CDC25A; CHEK2; CXorf15; CYP26A1; DCC1 (DSCC1); DTYMK; DNMT3A; EPHA; ETV4; FLJ20105 (ERCC6L); GPR19; HELLS; HESX1; MGC3101 (DBNDD1); PRO1853 (C2orf56); ISG20L1 (AEN); MCM10; NANOG; ORC1L; ORC2L; PWP2H; RBM14; RNU3IP2 (RRP9); SLD5 (GINS4); SLC5A6; and MYBL2) (68%) hESC-signature genes detected by the microarrays were significantly up-regulated in primary lung AdCa (FIG. 6), which represented an 89% overlap with the hESC-signature overexpressed in lung AdCa-xenografts. Strikingly, 12 of 15 (80%) BC-S hESC-signature genes, but only 6 of 25 (24%) remaining hESC-signature genes were significantly up-regulated in primary human lung AdCa (FIG. 6), thereby indicating that the BC-S hESC-signature genes predominantly contributed to the hESC-like molecular phenotype in lung AdCa.

### Example 12

This example demonstrates that a BC-S hESC-signature predicts aggressive clinical phenotype in lung adenocarcinoma.

High expression of the BC-S hESC-signature genes in lung AdCa determines a distinct, more aggressive clinical phenotype. The overall BC-S hESC-signature gene expression in 192 adenocarcinoma patients with known clinical information was determined using the BC-S hESC index, a cumulative measure of overexpression of 15 BC-S hESC-signature genes (calculated as a number of these genes whose expression was above the median in AdCa subjects). Among the 15 BC-S hESC-signature genes, 6 genes were identified (BRRN (NCAPH), DCC1 (DSCC1), DTYMK, FLJ20105 (ERCC6L), MCM10, and MYBL2), whose up-regulation in BC-S versus BC-NS was detected by both microarray and RNA-Seq analysis and whose expression in AdCa strongly correlated with the BC-S hESC index (rho>0.6, p<0.0001), representing, therefore, a cluster of co-expressed BC-S hESC-signature genes.

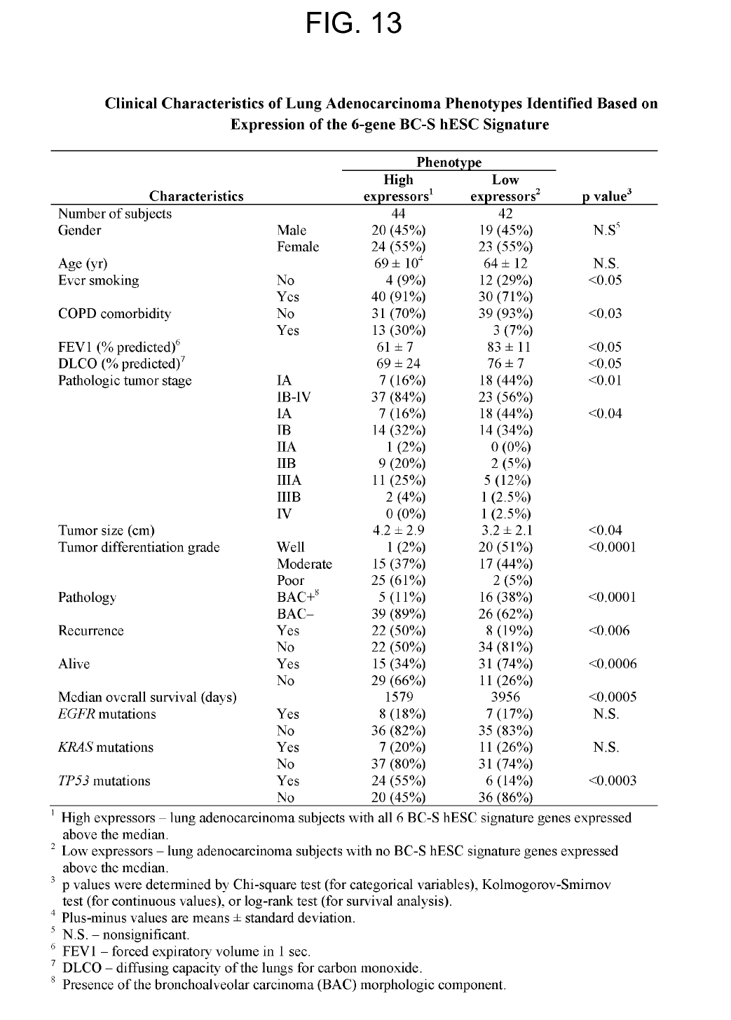

Based on the expression of these 6 BC-S hESC-signature genes, two groups of AdCa patients were identified: “high expressors” (all 6 genes expressed above the median level; n=44), and “low expressors” (all 6 genes expressed below the median; n=42). These two AdCa groups display strikingly opposite clinical and pathologic features (FIG. 13). Consistent with the smoking-dependent nature of the BC-S hESC-signature genes, 91% of high expressors were smokers versus 71% in the low expressor group (p<0.05). The high expressors exhibited higher comorbidity with chronic obstructive pulmonary disease (COPD) (p<0.03), lower lung function parameters such as forced expiratory volume in 1 sec (FEV1; p<0.05) and diffusing capacity of the lungs for carbon monoxide (DLCO; p<0.05).

However, most dramatic differences related to the tumor characteristics. Only 16% of high expressors, compared to 44% of low expressors, had a stage IA tumors (p<0.01), consistent with the overall tumor stage distribution analysis which revealed that high expressors have more advanced tumors (p<0.04). High expressors had larger tumor size (p<0.04), markedly poorer differentiation grade (p<0.0001) and lower frequency of the prognostically favourable bronchoalveolar carcinoma (BAC) (p<0.0001) than low expressors. Further, AdCa recurrence was observed in 50% of high expressors compared to 19% of low expressors (p<0.006). Strikingly, high expressors had markedly shorter overall median survival than the low expressors (1,579 days versus 3,956 days; p<0.0005 by log-rank test; FIG. 3E). Only 34% of high expressors versus 74% of low expressors were alive at the time of analysis (p<0.0006; FIG. 13). Together, these data suggest that high expression of the BC-S hESC-signature defines a distinct, more aggressive clinical phenotype of lung AdCa.

In summary, individuals with AdCa expressing the BC-S hESC-signature are predominantly smokers, have a higher co-morbidity with COPD and decreased lung function parameters FEV1 and DLCO, more advanced pathological stage, larger tumors, markedly poorer differentiation grade, higher recurrence frequency and, most strikingly, a 79-month shorter overall survival than lung AdCa patients not expressing this signature.

### Example 13

This example demonstrates that a BC-S hESC-signature is associated with the TP53-inactivation molecular phenotype.

AdCa subjects overexpressing highly co-expressed BC-S hESC genes were investigated for a distinct pattern of mutations. Although there was no significant difference in the frequency of mutations of EGFR or KRAS (FIG. 13), or STK11, BRAF, and PTEN (data not shown) between high and low expressors, AdCa subjects with high expression of the identified BC-S hESC-signature exhibited significantly higher frequency of mutations of the tumor suppressor gene TP53 (p<0.0002;FIG. 13).

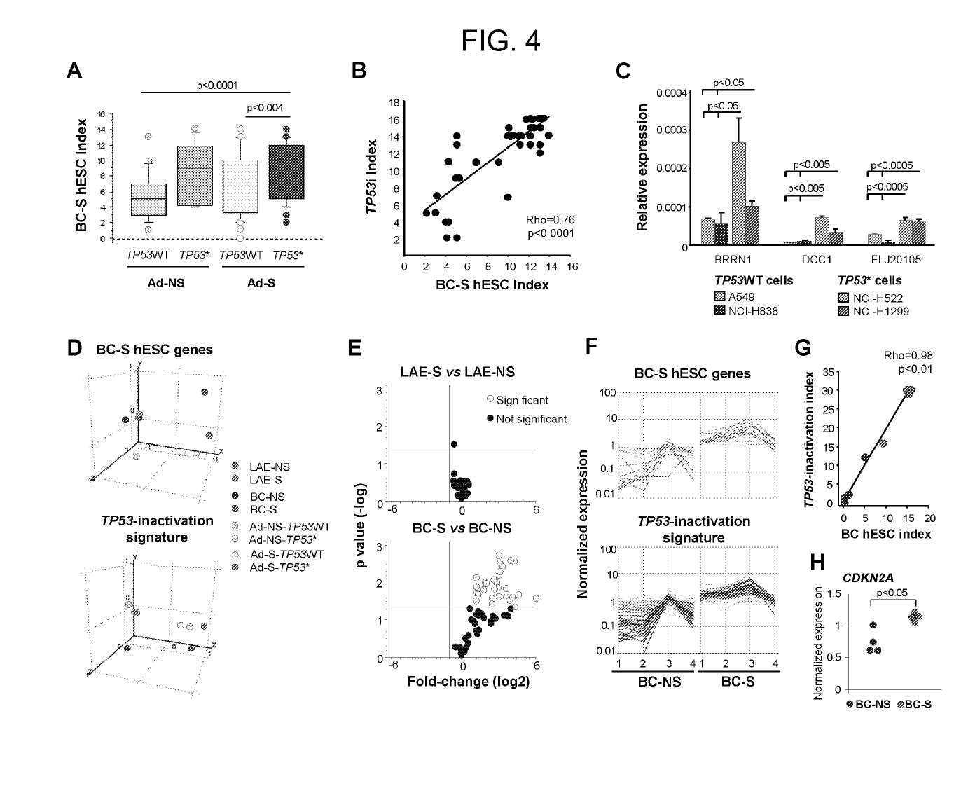

Consistently, analysis of the BC-S hESC index, i.e., a cumulative measure of the BC-S hESC-signature gene expression in AdCa subjects (calculated as the average number of BC-S hESC-signature genes expressed above the median level), revealed that the presence of TP53 mutations was associated with higher overall expression of BC-S hESC-signature genes (FIG. 4A). In AdCa-smokers with TP53 mutations, expression of the BC-S hESC-signature genes was strongly positively correlated with the expression of a subset of genes known to be up-regulated after TP53 mRNA silencing (Troester et al., BMC Cancer, 6: 276 (2006)) (referred to as the “TP53-inactivation signature,” rho=0.76; p<0.0001; FIG. 4B). Moreover, the NCI-H522 and NCI-HI299 lung carcinoma cell lines with TP53-inactivating mutations exhibit significantly higher expression of the BC-S hESC-signature genes BRRN/NCAPH, DCC1/DSCC1 and FLJ20105/ERCC6L than do both A549 and NCI-H838 TP53-wild-type lung cancer cell lines (FIG. 4C). This result indicates that the overexpression of BC-S hESC-signature genes in lung AdCa is associated with the molecular phenotype of TP53 inactivation.

Association of the BC-S hESC-signature overexpression in lung AdCa with TP53 mutations and with the molecular phenotype of TP53 inactivation suggests that the initial acquisition of the TP53 inactivation molecular phenotype could be present in the BC-S. To address this issue, a number of transcriptome analysis approaches were utilized.

First, PCA revealed that, based on the expression of the BC-S hESC-signature dataset, BC-S, but not BC-NS, shared a similar distribution as AdCa subjects with TP53 mutations (FIG. 4D, upper panel). Of note, BC-S distributed even more closely to the TP53 mutation-bearing AdCa, when the PCA was performed based on the TP53-inactivation signature dataset (FIG. 4D, lower panel), thereby indicating that BC-S and AdCa with TP53 mutations share a similar TP53-inactivation molecular pattern.

Second, the effect of smoking on expression of the TP53-inactivation signature genes in the healthy airway epithelium was directly analyzed. Similar to the hESC-signature genes (FIG. 2A), no significant difference was detected between the LAE-NS and LAE-S (FIG. 4E, upper panel), whereas there was a dramatic up-regulation of the TP53-inactivation signature genes in BC-S versus BC-NS (FIG. 4E, lower panel). This indicates that smoking selectively induces TP53-inactivation molecular phenotype in the basal cell population of the airway epithelium.

Third, it was investigated whether there is a correlation between the hESC-specific and TP53-inactivation signatures induced by smoking in airway basal cells. Indeed, expression patterns of these signatures turned out to be remarkably synchronous in basal cells of healthy individuals (FIG. 4F), and the overall expression of these signatures, determined as indexes, i.e., the number of genes expressed above the median in basal cell samples, strongly correlated (rho=0.98; p<0.01; FIG. 4G). Notably, there was significant up-regulation of CDKN2A in BC-S (FIG. 4H). Up-regulation of p14ARF encoded by CDKN2A has been linked to TP53 inactivation (Negrini et al., Nat. Rev. Mol. Cell Biol., 11: 220-228 (2010)). Together, these analyses provided transcriptome-based evidence that in AdCa and in BC-S, high expression of the BC-S hESC-signature genes is associated with the common molecular pattern of TP53-inactivation.

The observation that a significantly higher incidence of TP53 mutations in AdCa patients highly expressing the BC-S hESC-signature suggests two possible mechanistic models whereby smoking might reprogram airway basal cells toward a cells with lung cancer-relevant molecular phenotype.

As a first mechanism, TP53 inactivation might be required for acquisition of the hESC-like transcriptome phenotypes. TP53 is a tumor suppressor gene encoding phosphoprotein p53, which suppresses tumor formation by promoting apoptosis, activating cell cycle checkpoints, and inducing senescence (Yee et al., Carcinogenesis, 26: 1317-1322 (2005)). In addition to these classic functions, recent studies have documented a critical role for TP53 in maintaining embryonic stem cell genomic stability, inducing their differentiation (Lin et al., Nat. Cell. Biol., 7: 165-171 (2005)), and suppressing pluripotency (Hong et al., Nature, 460: 1132-1135 (2009); Kawamura et al., Nature, 460: 1140-1144 (2009); Li et al., Nature, 460: 1136-1139 (2009); Utikal et al., Nature, 460: 1145-1148 (2009)). TP53 mutations, a known biomarker of cigarette smoke exposure in lung cancer (Toyooka et al., Hum. Mutat., 21: 229-239 (2003)), represent the most common mutation in lung carcinomas, including SCC, AdCa, and SCLC, with a frequency varying between 40% and 75% depending on smoking status (Herbst et al., N. Engl. J. Med., 359: 1367-1380 (2008)).

Additionally, as described herein, different lung carcinoma cell lines harboring TP53 gene mutations overexpress hESC-signature genes with a pattern similar to that induced in BC-S, AdCa patients with TP53-mutations exhibited significantly higher expression of BC-S hESC-signature genes, and transcriptome analysis revealed a selective induction of genes associated with the TP53 inactivation in basal cells, but not in the complete airway epithelial population of healthy smokers. The molecular pattern of TP53 inactivation in BC-S was similar to that present in AdCa with TP53-mutations and the majority of SCC samples and hESC. Finally, overall expression levels of the hESC and TP53-inactivation signatures in airway basal cells strongly correlated.

Thus, it is possible that basal cells carrying inactivated TP53 acquire the hESC-like phenotype, gain a selective growth advantage, and eventually play a role in tumor initiation and propagation, thereby contributing to the development of poorly differentiated aggressive lung carcinomas. In support of this scenario, a widespread distribution of epithelial cells bearing a single point mutation in TP53 codon 245, a codon which is frequently mutated in lung cancer, has been detected in the airways of smokers without cancer (Franklin et al., J. Clin. Invest., 100: 2133-2137 (1997)), suggesting that a single clone of smoking-reprogrammed TP53-mutant progenitor cells might populate relatively large and distant areas of the airway epithelium prior to the formation of overt cancer. Furthermore, loss of heterozygocity at the TP53 locus and overexpression of the mutant p53 protein have previously been found in the dysplastic bronchial epithelium of smokers without lung cancer (Wistuba et al., J. Natl. Cancer Inst., 89: 1366-1373 (1997); Wistuba et al., Oncogene, 21: 7298-7306 (2002)). While not wishing to be bound by any particular theory as to the mechanism causing TP53 inactivation in the BC-S, epigenetic modifications that may occur in response to environmental factors, such as cigarette smoke, can repress gene function without changes in the DNA sequence (Sato et al., J. Thorac. Oncol., 2: 327-343 (2007)). Alternatively, DNA replication stress induced by cigarette smoking in proliferating basal cells might select for TP53 inactivation as a response to ongoing DNA damage (Negrini et al., Nat. Rev. Mol. Cell Biol., 11: 220-228 (2010)). Consistent with this concept, CHEK2, the central component of the DNA damage response (Reinhardt et al., Curr. Opin. Cell Biol., 21: 245-255 (2009)), was among the hESC-signature genes induced in BC-S.

As a second mechanism, TP53 mutations could be selected via oncogene-induced overexpression of p14ARF, which inhibits the murine double minute (MDM2), a protein that targets p53 for degradation (Zhang et al., Cell, 92: 725-734 (1998)). In favor of this model, CDKN2A, the gene which encodes p14ARF, was found to be significantly up-regulated in BC-S. When the function of p53 is lost, BC can escape its “genome guardian” functions and acquire the cancer-relevant hESC-like phenotype, and the precancerous lesion can become malignant. Indeed, DNA replication stress leading to genomic instability and selective pressure for p53 mutations has been described as an early mechanism of lung cancer development (Gorgoulis et al., Nature, 434: 907-913 (2005)).

### Example 4

This example demonstrates that a BC-S hESC-signature contributes to the hESC-like phenotype of various types of human lung cancer.

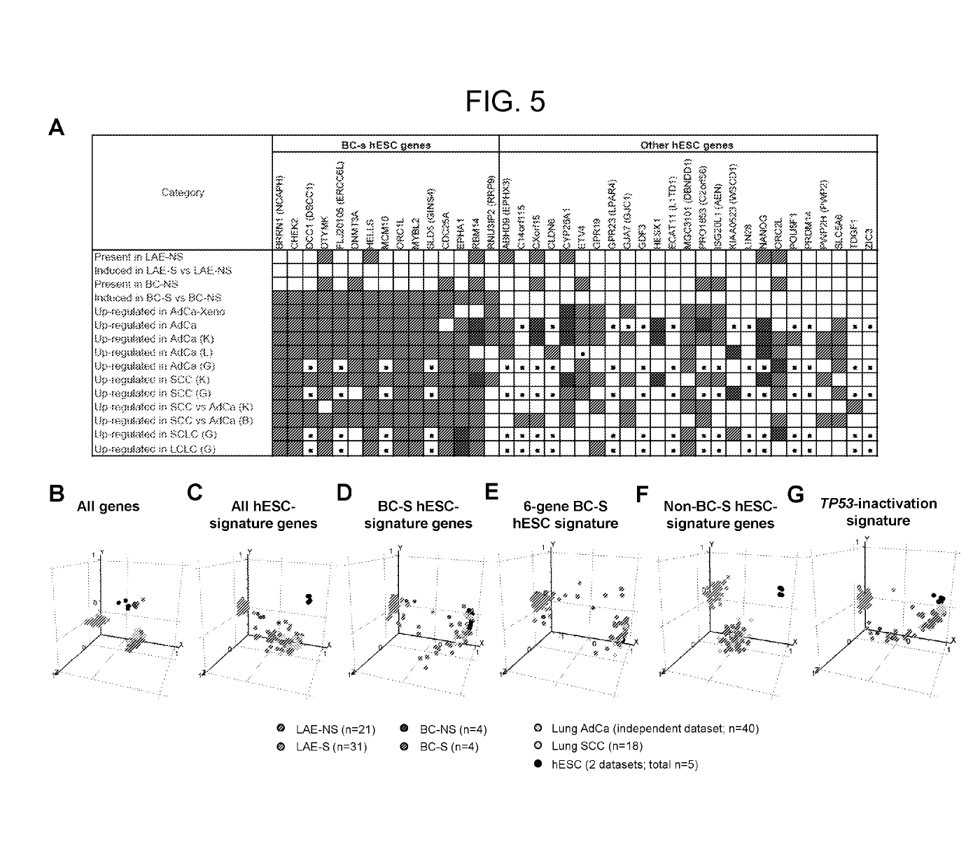

To validate the enrichment of BC-S hESC-signature genes in lung AdCa, three independent published AdCa datasets were analyzed (Garber et al., Proc. Natl. Acad. Sci. USA, 98: 13784-13789 (2001); Kuner et al., Lung Cancer, 63: 32-38 (2009); Landi et al., PLoS One, 3: e1651 (2008)). All three independent AdCa datasets revealed predominant up-regulation of the BC-S hESC-signature genes (FIG. 5A). Fourteen out of 15 (93%) BC-S hESC-signature genes versus 11 of 25 (44%) non-BC-S hESC-signature genes were up-regulated in the AdCa dataset of Kuner et al. (Kuner et al., Lung Cancer, 63: 32-38 (2009)); 13 of 15 (87%) BC-S hESC-signature genes versus 6 out 24 (25%) detectable other hESC-signature genes were up-regulated in the AdCa dataset of Landi et al (Landi et al., PLoS One, 3: e1651 (2008)); and 10 of 11 (91%) detectable BC-S hESC-signature genes versus 3 of 10 (30%) other hESC-signature genes were up-regulated in the AdCa dataset of Garber et al (Garber et al., Proc. Natl. Acad. Sci. USA, 98: 13784-13789 (2001)). There was remarkable overlap of overexpressed hESC-signature genes between these different datasets (FIG. 5A). Notably, non-BC-S hESC-signature genes CYP26A1, HESX1 and NANOG, associated with airway epithelial differentiation, were down-regulated in AdCa datasets (FIGS. 1D, 5A and 9).

Other types of human lung cancer also were investigated with respect to the up-regulation of the BC-S hESC-signature. In both analyzed lung squamous cell carcinoma (SCC) datasets (Garber et al., Proc. Natl. Acad. Sci. USA, 98: 13784-13789 (2001); Kuner et al., Lung Cancer, 63: 32-38 (2009)), overexpression of the BC-S hESC-signature genes was detected with a pattern surprisingly similar to that enriched in AdCa (FIG. 5A). Further analysis revealed that the overall expression level of the BC-S hESC-signature genes in lung SCC was considerably higher than in lung AdCa (FIG. 5A). The identical pattern of 8 out of 11 (73%) BC-S hESC-signature genes in the dataset of Garber et al (Garber et al., Proc. Natl. Acad. Sci. USA, 98: 13784-13789 (2001)) was up-regulated in the small cell lung carcinomas and large cell lung carcinomas versus only 2 of 10 (20%) non-BC-S hESC-signature genes up-regulated in these types of lung cancer. Strikingly, all 8 genes were among BC-S hESC-signature genes contributing to the hESC molecular phenotype of lung AdCa and SCC. Altogether, this data suggest that BC-S hESC-signature genes considerably contribute to the hESC-like molecular pattern of all major types of human lung cancer, thereby suggesting that reprogramming toward a hESC-like molecular phenotype in various types of lung cancer likely represents a common molecular process associated with the smoking-induced changes in the airway basal cell transcriptome.

Genome-wide PCA analysis revealed that, although both AdCa and SCC samples exhibited hESC-like features, airway basal cells from healthy individuals exhibited higher similarity to hESC with BC-S oriented closer to lung cancer samples (FIG. 5B). When the entire hESC-signature was used as an input dataset, a subset of AdCa samples and the majority of SCC shared with BC-S, but not BC-NS, similar distribution with a notable shift toward hESC (FIG. 5C). Further restriction of the analysis to the 15-gene BC-S hESC-signature revealed similarity of the SCC samples and a subset of the AdCa samples to both BC-S and hESC (FIG. 5D). This spatial pattern was effectively reproduced using the dataset containing 6 co-expressed prognostically relevant BC-S hESC-signature genes (FIG. 5E), but not the non-BC-S hESC-signature genes (FIG. 5F). Finally, SCC and a subset of AdCa samples clustered together with BC-S and hESC based on expression of the TP53-inactivation signature (FIG. 5G), thereby suggesting that acquisition of the transcriptome features of TP53 inactivation is coupled to the reprogramming toward a common hESC-like phenotype shared by BC-S and lung cancer.

In summary, there is remarkable overlap (up to 93%) between the BC-S hESC-signature genes and those overexpressed in the major types of human lung cancer, including lung AdCa, SCC, small cell lung carcinoma (SCLC), and large cell lung carcinoma (LCLC). In contrast, there is a relatively low contribution of other hESC-signature genes to the molecular phenotype of these carcinomas. Several themes relevant to the molecular and cellular origins of human lung cancer emerge from this observation.

Lung carcinomas result from a series of morphologic changes in the airway epithelium that evolve into distinct histological types (Wistuba et al., Ann. Rev Pathol., 1: 331-348 (2006)). Although smoking can cause all known types of lung cancer, SCC, and SCLC, which usually arise from the LAE, have a stronger association with smoking history than AdCa, which develops in the more distal airway epithelium (Herbst et al., N. Engl. J. Med., 359: 1367-1380 (2008)). Squamous dysplasia is a well-known precursor lesion of SCC (Auerbach et al., N. Engl. J. Med., 256: 97-104 (1957); Herbst et al., N. Engl. J. Med., 359: 1367-1380 (2008); Sato et al., J Thorac. Oncol., 2: 327-343 (2007); Wistuba et al., Oncogene, 21: 7298-7306 (2002); Wistuba et al., Ann. Rev Pathol., 1: 331-348 (2006)). Atypical adenomatous hyperplasia is considered a putative precursor lesion for AdCa, whereas neuroendocrine hyperplasia frequently precedes SCLC and a subset of large cell lung carcinoma (Herbst et al., N. Engl. J. Med., 359: 1367-1380 (2008); Sato et al., J Thorac. Oncol., 2: 327-343 (2007)). The molecular profiles associated with these cancers are also quite different, with EGFR mutations more common for AdCa in nonsmokers, KRAS mutations for AdCa in smokers, EGFR amplification in SCC, and MET overexpression in SCLC (Herbst et al., N. Engl. J. Med., 359: 1367-1380 (2008); Sato et al., J Thorac. Oncol., 2: 327-343 (2007); Wistuba et al., Ann. Rev Pathol., 1: 331-348 (2006)).

Airway basal cells have been regarded as putative cell-of-origin for SCC (Ooi et al., Cancer Res., 70: 6639-6648 (2010); Wistuba et al., Ann. Rev Pathol., 1: 331-348 (2006)), but not for other types of lung cancer. The remarkable similarity of the hESC-signature induced in BC-S to that overexpressed in 4 different types of human lung cancer suggests that reprogramming toward a hESC-like molecular phenotype in these types of lung cancer likely represents a common molecular process driven by smoking-induced changes in airway BC. In this context, selective smoking-induced activation of this hESC-signature gene expression pattern in BC-S might represent a common early pathogenetic event in the molecular evolution of these histologically distinct types of lung cancer. In support of this concept, certain genes typically overexpressed in SCC and AdCa are exclusively expressed in the airway basal cells within preneoplastic bronchial lesions (Smith et al., Oncogene, 22: 8677-8687 (2003); Smith et al., Br. J. Cancer, 91: 1515-1524 (2004)).

Activation of the hESC-like program in some carcinomas has been previously associated with their poor differentiation state and aggressiveness (Ben-Porath et al., Nat. Genet., 40: 499-507 (2008); Hassan et al., Clin. Cancer Res., 15(20): 6386-6390 (2009)). The data described herein, however, evidence that acquisition of the lung cancer-associated hESC-like molecular features associated with tumor aggressiveness begins in the airway basal cells of clinically healthy individuals chronically exposed to cigarette smoke. Expansion of the smoking-reprogrammed hESC-like basal cell clones in susceptible individuals provides a possible explanation for the progressive dedifferentiation associated with the development of smoking-associated lung carcinomas. In agreement with this model, patches of clonally-related cells harboring a uniform set of molecular alterations identical to those present in lung cancer have been found in the histologically normal airway epithelium of smokers without cancer (Park et al., J. Natl. Cancer Inst., 91: 1863-1868 (1999); Wistuba et al., J. Natl. Cancer Inst., 89: 1366-1373 (1997)), and the cells expressing basal cell markers CK5 and CK14 are predominant in SCC-related potentially preneoplastic lesions in smokers' airways (Ooi et al., Cancer Res., 70: 6639-6648 (2010)). In addition, although the basal cells utilized in the examples presented herein were from the LAE, the smoking-induced hESC-signature in these cells contributed to the molecular phenotype of both predominantly proximally-derived lung carcinomas such as SCC, SCLC, and LCLC, as well as AdCa, which is thought to originate in peripheral airways (Herbst et al., N. Engl. J. Med., 359: 1367-1380 (2008)). It is known that smoking creates a field of cancer-related molecular changes throughout the airway epithelium (Steiling et al., Cancer Prey. Res., 1: 396-403 (2008); Wistuba et al., Oncogene, 21: 7298-7306 (2002)). In support of this model, multiple clonal outgrowths of molecularly altered cells have been found widely distributed in the airway epithelium of smokers (Wistuba et al., J. Natl. Cancer Inst., 89: 1366-1373 (1997)), and smoking-induced changes in the LAE transcriptome have been used to predict lung cancers located at a distance from the sampled LAE (Spira et al., Proc Natl. Acad. Sci. USA, 101: 10143-10148 (2004)).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of detecting cancer, a progression of cancer, or a predisposition to cancer in a human, which method comprises
(a) obtaining a sample of airway basal cells from the human, and
(b) analyzing the sample to determine expression of one or more hESC-signature genes,
wherein the expression or lack of expression of the one or more hESC-signature genes is indicative of a presence or absence of cancer, a progression of cancer, or a predisposition to cancer in the human.

2. The method of claim 1, wherein the expression of the one or more hESC-signature genes in the sample is compared with expression of the one or more hESC-signature genes in a control.

3. The method of claim 2, wherein the control is a sample of airway basal cells obtained from the human at a previous time.

4. The method of claim 2, wherein the control is a sample of airway basal cells obtained from a human that does not have cancer.

5. The method of claim 2, wherein the control is a sample of airway basal cells obtained from a human that does not smoke.

6. The method of claim 2, wherein higher expression of the one or more hESC-signature genes in the sample compared to the expression of the one or more hESC-signature genes in the control is indicative of cancer, a progression of cancer, or a predisposition to cancer in the human.

7. The method of claim 6, wherein at least 2-fold higher expression of the one or more hESC-signature genes in the sample as compared to the expression of the one or more hESC-signature genes in the control is indicative of cancer, a progression of cancer, or a predisposition to cancer in the human.

8. The method of claim 1, wherein the one or more hESC-signature genes are selected from the group consisting of abhydrolase domain containing 9 (ABHD9) (EPHX3); barren homolog (Drosophila) (BRRN1) (NCAPH); cell division cycle 25A (CDC25A); CHK2 checkpoint homolog (S. pombe) (CHEK2); chromosome 14 open reading frame 115 (C14orf115); chromosome X open reading frame 15 (CXorf15); claudin 6 (CLDN6); cytochrome P450, family 26, subfamily A, polypeptide 1 (CYP26A1); defective in sister chromatid cohesion homolog 1 (S. cerevisiae) (DCC1) (DSCC1); deoxythymidylate kinase (thymidylate kinase) (DTYMK); DNA (cytosine-5-)-methyltransferase 3 alpha (DNMT3A); EPH receptor A1 (EPHA1); ets variant gene 4 (E1A enhancer binding protein, E1F) (ETV4); FLJ20105 protein (FLJ20105) (ERCC6L); G protein-coupled receptor 19 (GPR19); G protein-coupled receptor 23 (GPR23) (LPAR4); gap junction protein, alpha 7, 45 kDa (connexin 45) (GJA7) (GJC1); growth differentiation factor 3 (GDF3); helicase, lymphoid-specific (HELLS); homeo box (expressed in ES cells) 1 (HESX1); hypothetical protein FLJ10884 (ECAT11) (L1TD1); hypothetical protein MGC3101 (MGC3101) (DBNDD1); hypothetical protein PRO1853 (PRO1853) (C2orf56); interferon stimulated exonuclease gene 20 kDa-like 1 (ISG20L1) (AEN); KIAA0523 protein (KIAA0523) (WSCD1); lin-28 homolog (C. elegans) (LIN28); MCM10 minichromosome maintenance deficient 10 (S. cerevisiae) (MCM10); Nanog homeobox (NANOG); origin recognition complex, subunit 1-like (yeast) (ORC1L); origin recognition complex, subunit 2-like (yeast) (ORC2L); POU domain, class 5, transcription factor 1 (POU5F1); PR domain containing 14 (PRDM14); PWP2 periodic tryptophan protein homolog (yeast) (PWP2H); RNA binding motif protein 14 (RBM14); RNA, U3 small nucleolar interacting protein 2 (RNU3IP2) (RRP9); SLD5 homolog (SLD5) (GINS4); solute carrier family 5 (sodium-dependent vitamin transporter, member 6 (SLC5A6); teratocarcinoma-derived growth factor 1 (TDGF1); v-myb myeloblastosis viral oncogene homolog (avian)-like 2 (MYBL2); and zic family member 3 heterotaxy 1 (odd-paired homolog, Drosophila) (ZIC3).

9. The method of claim 8, wherein the one or more hESC-signature genes are selected from the group consisting of barren homolog (Drosophila) (BRRN1) (NCAPH);
cell division cycle 25A (CDC25A); CHK2 checkpoint homolog (S. pombe) (CHEK2);
defective in sister chromatid cohesion homolog 1 (S. cerevisiae) (DCC1) (DSCC1);
deoxythymidylate kinase (thymidylate kinase) (DTYMK); DNA (cytosine-5-)-methyltransferase 3 alpha (DNMT3A); EPH receptor A1 (EPHA1); FLJ20105 protein (FLJ20105) (ERCC6L); helicase, lymphoid-specific (HELLS); MCM10 minichromosome maintenance deficient 10 (S. cerevisiae) (MCM10); origin recognition complex, subunit 1-like (yeast) (ORC1L); RNA binding motif protein 14 (RBM14); RNA, U3 small nucleolar interacting protein 2 (RNU3IP2) (RRP9); SLD5 homolog (SLD5) (GINS4); and v-myb myeloblastosis viral oncogene homolog (avian)-like 2 (MYBL2).

10. The method of claim 1, wherein the cancer is lung cancer.

11. The method of claim 10, wherein the lung cancer is adenocarcinoma, squamous cell carcinoma, large cell carcinoma, or small cell carcinoma.

12. The method of claim 11, wherein the lung cancer has an aggressive clinical phenotype.

13. The method of claim 1, wherein the sample also has a mutated and/or inactivated of tumor suppressor gene TP53.

14. The method of claim 1, wherein the human is a smoker.

15. The method of claim 1, wherein the expression of the one or more hESC-signature genes is determined using microarray analysis, principle component analysis (PCA), and/or massive parallel RNA sequencing analysis (RNA-Seq).

16. An in vitro model for lung cancer, comprising airway basal cells that express one or more hESC-signature genes.

17. The model of claim 16, wherein the expression of the one or more hESC-signature genes is higher than expression of one or more hESC-signature genes in normal airway basal cells.

18. The model of claim 17, wherein the expression of the one or more hESC-signature genes is at least 2-fold higher than the expression of the one or more hESC-signature genes in the normal airway basal cells.

19. The model of claim 16, wherein the one or more hESC-signature genes are selected from the group consisting of abhydrolase domain containing 9 (ABHD9) (EPHX3); barren homolog (Drosophila) (BRRN1) (NCAPH); cell division cycle 25A (CDC25A); CHK2 checkpoint homolog (S. pombe) (CHEK2); chromosome 14 open reading frame 115 (C14orf115); chromosome X open reading frame 15 (CXorf15); claudin 6 (CLDN6); cytochrome P450, family 26, subfamily A, polypeptide 1 (CYP26A1); defective in sister chromatid cohesion homolog 1 (S. cerevisiae) (DCC1) (DSCC1); deoxythymidylate kinase (thymidylate kinase) (DTYMK); DNA (cytosine-5-)-methyltransferase 3 alpha (DNMT3A); EPH receptor A1 (EPHA1); ets variant gene 4 (E1A enhancer binding protein, E1AF) (ETV4); FLJ20105 protein (FLJ20105) (ERCC6L); G protein-coupled receptor 19 (GPR19); G protein-coupled receptor 23 (GPR23) (LPAR4); gap junction protein, alpha 7, 45 kDa (connexin 45) (GJA7) (GJC1); growth differentiation factor 3 (GDF3); helicase, lymphoid-specific (HELLS); homeo box (expressed in ES cells) 1 (HESX1); hypothetical protein FLJ10884 (ECAT11) (L1TD1); hypothetical protein MGC3101 (MGC3101) (DBNDD1); hypothetical protein PRO1853 (PRO1853) (C2orf56); interferon stimulated exonuclease gene 20 k Da-like 1 (ISG20L1) (AEN); KIAA0523 protein (KIAA0523) (WSCD1); lin-28 homolog (C. elegans) (LIN28); MCM10 minichromosome maintenance deficient 10 (S. cerevisiae) (MCM10); Nanog homeobox (NANOG); origin recognition complex, subunit 1-like (yeast) (ORC1L); origin recognition complex, subunit 2-like (yeast) (ORC2L); POU domain, class 5, transcription factor 1 (POU5F1); PR domain containing 14 (PRDM14); PWP2 periodic tryptophan protein homolog (yeast) (PWP2H); RNA binding motif protein 14 (RBM14); RNA, U3 small nucleolar interacting protein 2 (RNU3IP2) (RRP9); SLD5 homolog (SLD5) (GINS4); solute carrier family 5 (sodium-dependent vitamin transporter, member 6 (SLC5A6); teratocarcinoma-derived growth factor 1 (TDGF1); v-myb myeloblastosis viral oncogene homolog (avian)-like 2 (MYBL2); and zic family member 3 heterotaxy 1 (odd-paired homolog, Drosophila) (ZIC3).

20. The model of claim 19, wherein the one or more hESC-signature genes are selected from the group consisting of barren homolog (Drosophila) (BRRN1) (NCAPH); cell division cycle 25A (CDC25A); CHK2 checkpoint homolog (S. pombe) (CHEK2); defective in sister chromatid cohesion homolog 1 (S. cerevisiae) (DCC1) (DSCC1); deoxythymidylate kinase (thymidylate kinase) (DTYMK); DNA (cytosine-5-)-methyltransferase 3 alpha (DNMT3A); EPH receptor A1 (EPHA1); FLJ20105 protein (FLJ20105) (ERCC6L); helicase, lymphoid-specific (HELLS); MCM10 minichromosome maintenance deficient 10 (S. cerevisiae) (MCM10); origin recognition complex, subunit 1-like (yeast) (ORC1L); RNA binding motif protein 14 (RBM14); RNA, U3 small nucleolar interacting protein 2 (RNU3IP2) (RRP9); SLD5 homolog (SLD5) (GINS4); and v-myb myeloblastosis viral oncogene homolog (avian)-like 2 (MYBL2).

21. The model of claim 16, wherein the expression of the one or more hESC-signature genes is induced with smoke or smoke extract.
